(19) United States
(12) Patent Application Publication
(10) Pub. No.: US 2011/0212179 A1
(43) Pub. Date: Sep. 1, 2011

(54) MICRO-SPHERICAL POROUS BIOCOMPATIBLE SCAFFOLDS AND METHODS AND APPARATUS FOR FABRICATING SAME

(76) Inventor: David Liu, Point Roberts, WA (US)

(21) Appl. No.: 13/126,456

(22) PCT Filed: Oct. 30, 2009
(86) PCT No.: PCT/US2009/062744

§ 371 Date: Apr. 27, 2011

Related U.S. Application Data
(60) Provisional application No. 61/197,803, filed on Oct. 30, 2008.

(51) Int. Cl. A61K 9/14; A61K 35/00; A61K 35/12; C12N 5/071; B01J 19/00; C08G 63/91; C08F 20/56; C08F 116/06; C08J 9/00; C08G 73/10; B82Y 30/00

(52) U.S. Cl. 424/489; 424/93.1; 424/93.7; 435/325; 422/243; 521/189; 521/149; 521/141; 521/92; 521/84.1; 521/89; 521/134; 521/183; 977/773

(57) ABSTRACT

Provided herein are bimodal porous polymer microspheres comprising macropores and micropores. Also provided herein are methods and apparatus for fabrication such microspheres. Further provided herein are methods of using bimodal porous polymer microspheres.

### CROSS-REFERENCE TO RELATED APPLICATIONS

This application claims the benefit of U.S. Provisional Ser. No. 61/197,803 filed Oct. 30, 2008, which is herein incorporated by reference in its entirety.

### FIELD

Provided herein are bimodal porous polymer microspheres comprising macropores and micropores, and methods and apparatus for fabrication of such microspheres. Further provided herein are methods of using bimodal porous polymer microspheres.

### BACKGROUND

Synthetic biocompatible porous scaffolds, such as those disclosed in U.S. Pat. No. 6,337,198 (Levene et al.) may be used as frameworks for supporting cell growth and tissue regeneration. Levene et al. disclose a method for fabricating a polymer-based scaffold with a bimodal pore distribution, where the larger pores are in the range of about 50 to about 500 microns and the smaller pores are less than 20 microns. One drawback of the Levene et al. scaffold structure is its relatively large overall size. The scaffolds disclosed in Levene et al. are fabricated using a mold (or dish) that forms a continuous polymer superstructure having the shape of the dish. Levene et al. describe a dish of 8 mm in diameter and 2-3 mm thick. Scaffolds of this size must be surgically implanted at the target site and are not suitable for injection through catheter, needle or tubing, or for delivery to smaller environments within the body such as vascular environments.

Solid biocompatible spheres (without pores) are known in the prior art as a means of deliberately blocking blood flow to targeted tissues (such as cancerous tissues) in order to counteract the growth of such tissues. Such techniques are known as embolization or embolotherapy—see Liu et al., JVIR 2005, 16(7):911-935 and Chua et al., Clinical Radiology 2005, 60(1):116-122. These solid spheres may also deliver radiation to provide radiation therapy to targeted tissues—see Salem et al., JVIR 2006, 17(8):1251-1278. However, complete embolization in the target area may not be desirable since blood flow is required to provide oxygen.

Thus, there is a general need for porous scaffolding structures such as microspheres of a smaller size, suitable for injection through a catheter, needle or tubing, suitable for delivery to and suspension in microscopic environments within a living organism (in vivo), and capable of providing continued blood flow with minimal or optimized embolic effect.

### SUMMARY

The following embodiments and aspects thereof are described and illustrated in conjunction with systems, tools and methods which are meant to be exemplary and illustrative, not limiting in scope. Other limitations of the related art will become apparent to those of skill in the art upon a reading of the specification and a study of the drawings. In various embodiments, one or more of the above-described problems have been reduced or eliminated, while other embodiments are directed to other improvements.

In one aspect, provide herein is a method for preparing a bimodal porous polymer particle, said method comprising: (a) providing a homogeneous solution comprising a base polymer, a first solvent and a second solvent; (b) adding a macropore spacer material to the solution; (c) injecting droplets of the solution into a quenching device; (d) quenching droplets of the solution to solidify the base polymer into particles having macropores and micropores; (e) extracting substantially spherical particles from the quenching device, and optionally sieving the particles; and (f) optionally washing the macropore spacer material from the particles. In some embodiments of the method, one or more of (a)-(f) is carried out at a temperature of less than 42° C. In one embodiment of the method, each of (a)-(f) are carried out at a temperature of less than 42° C. Also provided herein are particles made by these methods. In some embodiments, the particle is a microsphere, such as a substantially spherical microsphere. In other embodiments, the particle further comprises an additive.

In a second aspect, provided herein is a bimodal porous particle comprising a base polymer, wherein the particle comprises macropores having a diameter ranging from about 20 to about 500 microns and micropores having a diameter ranging from about 1 to about 70 microns, and wherein the microspheres have a diameter ranging from about 50 to about 1100 microns. In some embodiments, the particle is a microsphere, such as a substantially spherical microsphere. In other embodiments, the particle further comprises an additive. In other embodiments, the particle further comprises a cell.

In a third aspect, provided herein is a method of complete or partial embolization in a patient, comprising administering to the patient the bimodal porous particle provided herein. In some embodiments, the particle provides or allows temporary or continued perfusion of the blood vessel, vein, artery, tissue or organ, e.g., about 5%, about 10%, about 20%, about 30%, about 40%, about 50%, about 60%, about 70%, about 80%, about 90%, or about 95% as compared to the amount of perfusion in the absence of particle.

In a fourth aspect, provided herein is a method of treating or otherwise managing a cancer or tumor, or a symptom thereof, in a patient, comprising administering a bimodal porous particle provided herein to a blood vessel, vein or artery of the patient that directly or indirectly supplies the cancer or tumor with blood. In certain embodiments, the particle provides or allows temporary or continued perfusion of the cancer or tumor.

In a fifth aspect, provided herein is a method for delivering cells to a patient, comprising administering a bimodal porous particle provided herein to the patient, wherein the bimodal porous particle comprises cells. In certain embodiments, the cells are delivered to or otherwise contacted with a tissue or organ of the patient. In certain embodiments, the particle provides or allows temporary or continued perfusion of the area where the cell is delivered.

In a sixth aspect, provided herein is a method for retaining cells in a tissue or organ of a patient, comprising administering a bimodal porous particle provided herein to the patient, wherein the bimodal porous particle comprises cells, and contacting the cells with the tissue or organ. In certain embodiments, the particle provides or allows temporary or continued perfusion of the area where the cell is delivered.

In a seventh aspect, provided herein is a method for engrafting cells in a tissue or organ of a patient, comprising administering a bimodal porous particle provided herein to the patient, wherein the bimodal porous particle comprises cells, and contacting the cells with the tissue or organ. In certain embodiments, the particle provides or allows temporary or continued perfusion of the area where the cell is delivered.

In an eighth aspect, provided herein is a method for tissue regeneration in a patient, comprising administering a bimodal porous particle provided herein to the patient, wherein the bimodal porous particle comprises cells, wherein the cells are contacted with the tissue being regenerated. In certain embodiments, the tissue is heart, lung, nervous, brain, liver or pancreas tissue. In other embodiments, the tissue is a blood vessel, vein or artery. In some embodiments, the tissue is a wound or other injured tissue. In certain embodiments, the particle provides or allows temporary or continued perfusion of the area where the cell is delivered. In certain embodiments, the particle is administered to the heart, lung, nervous system, brain, lung, liver or pancreas of the patient.

In a ninth aspect, provided herein is a method for islet cell transplantation in a patient, comprising administering a bimodal porous particle provided herein to the patient, wherein the cell is an islet cell. In certain embodiments, the particle provides or allows temporary or continued perfusion of the area where the cell is delivered.

In a tenth aspect, provided herein is a method for treating or otherwise managing diabetes, or a symptom thereof, in a patient, comprising administering a bimodal porous particle provided herein to the patient, wherein the cell is an islet cell. In some embodiments, the islet cell produces insulin. In certain embodiments, the particle is administered to the liver (e.g., a portal vein of the liver) of the patient. In certain embodiments, the particle provides or allows temporary or continued perfusion of the area where the cell is delivered.

In an eleventh aspect, provided herein is a method for intraarterial brachytherapy in a patient, administering a bimodal porous particle provided herein to the patient. In some embodiments, the particle comprises an additive, such as a radioactive material. In other embodiments, the particle is administered to a cancer or tumor. In certain embodiments, the particle provides or allows temporary or continued perfusion of the cancer or tumor. In some embodiments, the particle completely or partially embolizes a blood vessel, vein or artery of the patient that directly or indirectly supplies the cancer or tumor with blood.

In a twelfth aspect, provided herein is a method for delivering an additive to a patient, comprising administering a bimodal porous particle provided herein to the patient, wherein the particle comprises the additive. In some embodiments, the additive is a therapeutic agent or drug. In other embodiments, the additive is a tracer or imaging agent. In other embodiments, the additive is a diagnostic agent.

In a thirteenth aspect, provided herein is a method of prolonged and/or controlled delivery of an additive (e.g., a therapeutic agent or drug) in a patent, comprising administering a bimodal porous particle provided herein to the patient, wherein the particle comprises the additive. In certain embodiments, the particle is administered to the patient by intraluminal, interstitial, subdermal, transdermal or subcutaneous administration.

In a fourteenth aspect, provided herein is a method of trapping, filtering or extracting cells from the blood of a patient, comprising administering a bimodal porous particle provided herein to the patient, wherein the particle allows for perfusion through the particle, and wherein the particle traps, filters, attracts, promotes cell migration, or extracts the cells from the blood or adjacent tissue of the patient.

In a fifteenth aspect, provided herein is an apparatus for fabricating bimodal porous polymer particles, comprising: (a) a storage vessel; (b) a quenching tower; (c) an injector comprising a nozzle, wherein the nozzle has a diameter ranging from about 5 to about 1100 microns in diameter; and wherein the vessel is connected to the injector; and (d) one or more microsieves. In certain embodiments, the nozzle can be substituted by other means of providing laminar flow through an aperature, such as in established focused fluidics techniques. In one embodiment, a flow-focusing geometry integrated into a plannar microchannel can be used to substitute the nozzle. In such embodiment, both monodisperse and polydisperse droplets can be produced. See Anna et al., (2003) Appl. Phys. Lett. 82, 364. In certain embodiments, the injector further comprises a chamber and a piston. In other embodiments, the storage vessel further comprises an agitator or mixer. In some embodiments, the storage vessel comprises a first solvent, a second solvent and a base polymer provided herein. In other embodiments, the apparatus further comprising a conduit attached to the storage vessel, wherein the conduit optionally comprises a micropore spacer material provided herein.

In addition to the exemplary aspects and embodiments described above, further aspects and embodiments will become apparent by reference to the drawings and by study of the following detailed descriptions.

### Terminology

Unless defined otherwise, all technical and scientific terms used herein have the same meaning as is commonly understood by one of ordinary skill in the art. All patents, applications, published applications and other publications are incorporated herein by reference in their entirety. In the event that there is a plurality of definitions for a term herein, those in this section prevail unless stated otherwise.

The term “about” or “approximately” means within 20%, within 10%, within 5%, or within 1% or less of a given value or range.

The term “additive” refers to a substance, molecule or material (e.g., bio-active material) a microsphere may carry, contain, be impregnated with, coated with, or bonded with. In some embodiments, an additive may be added to the base polymer during fabrication process. Non-limiting examples of additives include therapeutic agents, cells, cell differentiating and signaling materials, cell adhesion factors or promoters (e.g., selectins, collagen, gelatin, glucosaminoglycans, fibronectins, lectins, polycations, polylysine, chitosan and the like, or any other natural or synthetic biological cell adhesion agent), antibodies, blood clotting or anti-clotting agents, radioactive sources and chemotherapy materials.

As used herein, “administer,” “administration” and “administering” refers to the act of injecting or otherwise physically delivering a substance as it exists outside the body (e.g., la particle or microsphere provided herein) into a patient, such as by, but not limited to, pulmonary (e.g., inhalation), mucosal (e.g., intranasal), intradermal, intravenous, intraarterial, intrabiliary, intraocular, intraosseous, intramuscular delivery and/or any other method of physical delivery described herein or known in the art. When a disease, or a symptom thereof, is being treated or otherwise managed, administration of the substance typically occurs after the onset of the disease or symptoms thereof. When a disease, or symptom thereof, is being prevented, administration of the substance typically occurs before the onset of the disease or symptoms thereof. Such administration, in certain embodiments, results in the delivered particles (e.g., a particle and/or additive provided herein) contacting the target area (e.g., a tissue or organ).

The terms “bimodal pore distribution,” “bimodal pore size distribution” and “bimodal pore size” are used interchangeably and refer to two different ranges of pore sizes (e.g., macropores versus micropores or large pores versus small pores) present in the porous polymer scaffolds or microspheres provided herein. For instance, in some embodiments, the size of the macropores in the bimodal pore distribution can be on the order of about 20 to about 500 microns and the micropores can be on the order of about 1 to about 70 microns. In other embodiments, the size of the macropores can be on the order of about 20 to about 200 microns and the micropores can be on the order of about 1 to about 40 microns. The term “bimodal porous microspheres,” “bimodal porous polymeric microspheres” or “bimodal porous polymer microspheres” as used herein refers to polymeric microspheres comprising pores of bimodal size distribution.

As used herein, the term “bioabsorbable” refers to the ability of a material to degrade, be metabolized by the body in vivo or be eliminated from the body.

The term “biodegradable” as used herein refers to a material or object (e.g., polymer, microsphere) that is capable of being absorbed by the body, chemically, physiologically, or by other biological means, over a period of time.

The term “biocompatible” as used herein refers to the property or ability of a material or object (e.g., polymer or microspheres) to be applied in vivo (e.g., to cells, tissues or organs) without eliciting significant immune responses, inflammation or other adverse responses unless otherwise intended.

“Cell adhesion promoter” as used herein means any material that, because of their presence in or association with the microspheres, promotes or enhances the adhesiveness of cells to the surface of the microspheres. These materials are often proteins that are bound to the surface of the microspheres through covalent bonds of the proteins and the polymers.

The term “effective amount” as used herein refers to the amount of a therapy (e.g., a microsphere or composition provided herein) which is sufficient to reduce and/or ameliorate the severity and/or duration of a given disease and/or a symptom related thereto. In certain embodiments of the methods provided herein, an effective amount of the particle is administered to the patient.

The term “engraftment” as used herein refers to a process by which transplanted cells are accepted by a host tissue, survive and persist in that environment, e.g., for a period of 24 hours or more. In certain embodiments, the transplanted stem cells further reproduce.

The term “in combination” as used herein in the context of the administration of other therapies refers to the use of more than one therapy. The use of the term “in combination” does not restrict the order in which therapies are administered to a subject. A first therapy can be administered before (e.g., 1 minute, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a given disease. Any additional therapy can be administered in any order with the other additional therapies. In certain embodiments, the particles provided herein can be administered in combination with one or more therapies (e.g., therapies that are not the magnetically labeled cells that are currently administered to prevent, treat, manage, and/or ameliorate a given disease or other symptom related thereto). Non-limiting examples of therapies that can be administered in combination with the particles provided herein include additives, such as analgesic agents, anesthetic agents, antibiotics, or immunomodulatory agents or any other agent listed in the U.S. Pharmacopoeia and/or Physician's Desk Reference.

A used herein, “injectable” means capable of being administered, delivered or carried into the body via syringe, catheters, needles or other means for injecting or infusing the microspheres in a liquid medium. In certain embodiments, the particles provided herein are injectable particles.

As used herein, the terms “manage,” “managing,” and “management” refer to the beneficial effects that a subject derives from a therapy (e.g., microspheres provided herein), which does not result in a cure of the infection. In certain embodiments of the methods provided herein, a subject is administered one or more therapies to “manage” a given disease or one or more symptoms related thereto, so as to prevent the progression or worsening of the disease.

As used herein, the term “microspheres” refer to a polymer or combinations of polymers made into bodies of various sizes. The microspheres as used herein can be in any shape, although they are often in substantially spherical shape. These structures of the microspheres may be generally spherical or spheroid in shape or bounded by imaginary spherical or spheroid shapes. The microspheres may be sterilized by any method known in the art, for example, by irradiation, such as gamma or beta irradiation. The microspheres provided herein may comprise other materials as described and defined herein. However, it will be appreciated, that the term “microsphere” represents a convenient description for the purposes of explanation of the compositions and methods provided herein, and that, in certain embodiments, the exemplary microspheres described herein are not necessarily limited to being precisely spherical in shape (e.g., are particles).

The term “pharmaceutically acceptable” as used herein means being approved by a regulatory agency of the Federal or a state government, or listed in the U.S. Pharmacopeia, European Pharmacopeia or other generally recognized pharmacopeia for use in animals, and more particularly in humans

The term “polymer” refers to a molecule consisting of multiple repetition of molecular units. The polymer as used herein can be in any form of structure, for instance, linear or branched (e.g., a “multi-arm” or “star-shaped). Accordingly, the term “base polymer” refers to a polymer which may be incorporated into a composition comprising, for example, a polymer and one or more additives. The term “copolymer” refers to a polymer formed by a combination of two or more monomeric or polymeric species. The term “block copolymer” refers to a copolymer composed of block macromolecules. In certain embodiments, adjacent blocks in a block copolymer comprise units derived from different species of monomer or from the same species of monomer but with a different composition or sequence distribution of constitutional units. It is noted that the term “polymer” as used herein should not be limited to polymers in the strict chemical sense and that other suitable materials having the characteristics described herein may be used.

As used herein, the terms “prevent,” “preventing,” and “prevention” refer to the total or partial inhibition of a given disease; the total or partial inhibition of the development or onset of disease progression of given disease, or a symptom related thereto in a subject; the total or partial inhibition of the progression of an given disease or a symptom related thereto.

The terms “regenerate,” “regeneration” and “regenerating” as used herein in the context of tissue or organ regeneration refer to the process of growing and/or developing new tissue. In certain embodiments, tissue regeneration comprises activation and/or enhancement of cell proliferation. In other embodiments, tissue regeneration comprises activation and/or enhancement of cell migration.

The term “retention” as used herein refers to a process by which transplanted cells are retained by a host tissue or organ, e.g., are accepted, survive and persist in that environment, e.g., for a period of minutes to hours. In certain embodiments, the transplanted cells further reproduce.

The term “stem cells” refers to cells that have the capacity to self-renew and to generate differentiated progeny. In certain embodiments, the stem cells are mesenchymal stem cells.

As used herein, the terms “subject” and “patient” are used interchangeably. As used herein, a subject is a mammal such as a non-primate (e.g., cows, pigs, horses, cats, dogs, rats, rabbits, etc.) or a primate (e.g., monkey and human) comprising administration of particles as provided herein. In some embodiments, the patient is in need of treatment or management of the disease or symptom thereof. In specific embodiments, the subject is a human.

As used herein, the term “substantially spherical” or “generally spherical” refers to a shape that is close to a perfect sphere, which is defined as a volume that presents the lowest external surface area. Specifically, “substantially spherical” as used herein means, when viewing any cross-section of the particle, the difference between the average major diameter and the average minor diameter is less than 20%. In some embodiments, the surfaces of the microspheres provided herein appear smooth under magnification of up to 1000 times.

The terms “therapeutic agent” or “therapeutic drug” can be used interchangeably herein and refers to any therapeutically active substance that is delivered to a bodily conduit of a living being to produce a desired, usually beneficial, effect.

As used herein, the term “therapy” refers to any protocol, method and/or agent that can be used in the management, treatment and/or amelioration of a given disease, or a symptom related thereto. In certain embodiments, the terms “therapies” and “therapy” refer to a biological therapy, supportive therapy, and/or other therapies known to one of skill in the art, such as medical personnel, useful in the management or treatment of a given disease, or symptom related thereto.

“Tissue construction,” “tissue generation,” “tissue engineering” and “tissue repair,” are used interchangeably in the context of the compositions and methods provided herein and refer to the processes or events associated with the healing, growth, regrowth, or change of conditions of tissues. The tissues encompassed include, but are not limited to, muscle tissues, connective tissues, fats, and, nerve tissues. The tissue defects suitable for the treatment and management methods provided herein include, but not limited to, defects in a patient's heart, coronary vessels, blood vessels, spinal cord, bone, cartilage, tendon, ligament, breast, liver, gallbladder, bile duct, pancreas, intestinal tissues, urinary system, skin, hernia, and dental tissues.

As used herein, the terms “treat,” “treatment” and “treating” refer to the reduction or amelioration of the progression, severity, and/or duration of a disease or a symptom thereof.

### BRIEF DESCRIPTION OF DRAWINGS

Exemplary embodiments are illustrated in referenced figures of the drawings. It is intended that the embodiments and figures disclosed herein are to be considered illustrative rather than restrictive.

FIGS. 1A-1B depict an exemplary biocompatible porous microsphere (microsphere 20) according to particular embodiments provided herein. FIG. 1A is a cross-sectional view of the biocompatible porous microsphere 20. FIG. 1B depicts a magnified view of a portion of a cross-section of microsphere 20, illustrating its bimodal pore distribution.

FIG. 2 shows an apparatus (apparatus 50) for fabricating biocompatible porous microspheres (e.g., microsphere 20) according to a particular embodiment provided herein.

FIG. 3 is a flowchart illustrating the steps of an exemplary method (method 200) of fabricating biocompatible porous microspheres (e.g., microsphere 20) according to a particular embodiment provided herein.

### DETAILED DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

### A. Bimodal Porous Microspheres

Provided herein are biocompatible bimodal porous scaffolding structures (e.g., particles, such a microspheres) of variable sizes for use in a wide range medical applications. In certain embodiments, the scaffold structures are fabricated using the surface tension of liquid material to provide generally spherical or spheroid shapes or to be bounded by imaginary spherical or spheroid shapes and are referred to herein as microspheres.

In some embodiments, the microspheres are in a range of about 50 to about 1100 microns in diameter. In other embodiments, the microspheres are under about 500 microns in diameter. In still other embodiments, the microspheres are under about 300 microns in diameter. These diameters permit the microspheres to be delivered to target tissues in vivo via catheter, needle, tubing, or the like by various pathways including vascular, intraductal, transesophogeal, subcutaneous, subdermal, submucosal, transbronchial, or interstitial. In some embodiments, the size of the macropores in the bimodal pore distribution may be on the order of about 20 to about 500 microns and the micropores may be on the order of about 1 to about 70 microns. In other embodiments, the size of the macropores may be on the order of about 20 to about 200 microns and the micropores may be on the order of about 1 to about 40 microns. In certain embodiments, the bimodal porous polymer microspheres further comprise an additive.

In one embodiment, provided herein is a bimodal porous polymer microsphere comprising macropores having a diameter ranging from about 20 to about 500 microns, or from about 20 to about 200 microns; and micropores having a diameter ranging from about 1 to about 70 microns or from about 1 to about 40 microns. In certain embodiments, the wherein the microspheres have a diameter ranging from about 50 to about 1100 microns, from about 50 to about 500 microns, or from about 50 to about 300 microns.

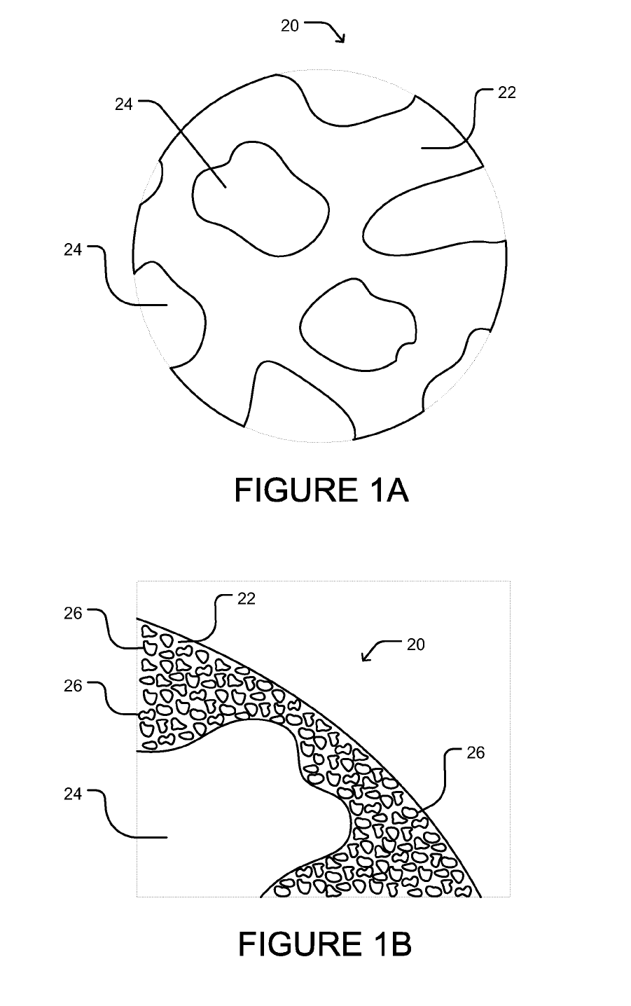

FIG. 1A depicts an exemplary biocompatible porous microsphere 20 according to particular embodiments provided herein, as viewed from a cross-section taken through its center. FIG. 1B depicts a magnified view of a portion of a cross-section of microsphere 20, illustrating its bimodal pore distribution.

Microsphere 20 comprises a biocompatible base polymer or monomer 22 (referred to in this description as base polymer 22). In particular embodiments, base polymer 22 may also be bioabsorbable and/or biodegradable. Examples of suitable base polymers 22 are described elsewhere herein.

While the bimodal porous microspheres (microsphere 20) provided herein may be constructed to have any desired size, in certain embodiments, the size of microsphere is in the range of about 50 to about 1100 microns in diameter to facilitate catheter-based delivery of microspheres 20 into target tissue beds by various pathways including vascular (arterial, venous, portal), intraductal (e.g. biliary tree), transesophageal, subcutaneous, subdermal, submucosal, transbronchial, or interstitial delivery. Other possible delivery mechanisms include injection via a needle or tubing and direct placement in, on or in a vicinity of the target tissue. Other non-limiting examples of delivering microspheres are provided elsewhere herein.

Microspheres provided herein may be permeated with pores of various sizes and shapes. In the illustrated example of FIGS. 1A and 1B, microsphere (20) has a bimodal pore distribution, which includes relatively large pores (macropores) (24) and relatively small pores (micropores) (26). In particular embodiments, micropores have diameters in a range of about 1 to about 70 microns and macropores have diameters in a range of about 20 to about 500 microns. Such a bimodal pore distribution, in comparison to a uniform pore distribution, tends to increase the overall porosity and surface area of microspheres, reduce the mass density of microspheres, and increase the number of interconnections between the pores in microspheres. Bimodal pore distribution may permit improved blood flow dynamics through and around microspheres in vivo, reducing blood flow resistance, turbulence and pressure differentials caused by microspheres and thereby reducing perfusional gradients and the potential formation of blood clots (thrombogenesis). Similar improved flow dynamics may be produced in fluids other than blood. Bimodal distribution may also allow for site of sequestration of cells, enabling adhesion, fixation or differentiation in the lager pore, while maintaining perfusion through the microsphere via either macropores or micropores.

Variations in size of macropores and micropores allow for delivery, capture or retention of a wide variety of bio-active materials (such as cells of varying sizes) at target tissues. The increased surface area and interconnectivity of bimodal pore distributions may also facilitate cell growth, tissue regeneration, vascularization, and delivery of higher concentrations of bio-active materials to target tissues. Macropores may provide sufficiently open space for the formation of functional tissue within the scaffold of microspheres while micropores can form channels between macropores to increase or otherwise optimize cell-to-cell contact or communication, diffusion of nutrients and oxygen to the cells, osmosis, and surface patterning to guide the cells. Macropores may also serve as channels through which blood or other fluids may flow, and may potentially differentiate into a permanent conduit (such as an artificial blood vessel, bile duct or vein, for example). The porosity of the bimodal pore distribution in particular microspheres may be designed to allow the specific gravity of microspheres to closely match (or to otherwise have a certain relationship (e.g., heavier or lighter)) than that of its target fluid suspension or target tissues.

In one embodiment, the particles further comprise a cell adhesion promoter. In another embodiment, the particles comprises a gelatin. In some embodiments, the particles are crosslinked. In other embodiments, the particles are not crosslinked. In certain embodiments, the particles are sterile.

The microspheres provided herein may comprise, in addition to the particles, other materials. In some embodiments, macropores and micropores of the microspheres provided herein can comprise, e.g., carry, contain, be impregnated with, coated with, or bonded with, various bioactive materials or other additives provided herein such as, but not limited to, therapeutic agents, cells, cell differentiating and signaling materials, cell adhesion factors (e.g., selectins), antibodies, blood clotting or anti-clotting agents, and chemotherapy materials. Other non-limiting examples of additives are provided elsewhere herein. Such material-carrying microspheres allow for the delivery and prolonged exposure of a selected therapy to a specific target tissue. The sizes of macropores and micropores may be varied according to embodiments provided herein to allow for the carrying of additives of varying sizes.

### B. Apparatus and Methods of Fabricating Bimodal Porous Microspheres

Also provided herein are exemplary apparatus for and methods of fabricating a biocompatible porous particle (e.g., a microsphere) provided herein.

Parts of the basic chemistry for fabricating a scaffolding comprising a base polymer with a bimodal pore distribution comprising macropores and micropores is described in U.S. Pat. No. 6,337,198 (Levene et al.), which is hereby incorporated herein by reference in its entirety. In certain embodiments, the process involves preparing a homogeneous solution comprising a base polymer dissolved in a first solvent (in which the base polymer is soluble) and a second solvent in which the base polymer is insoluble, but which is miscible in the first solvent. According to Levene et al., the homogeneous solution thus prepared is cast in a mold atop solid macropore spacer particles having suitable sizes. According Levene et al., the macropore spacer particles are not soluble in the first solvent, but may be water-soluble. The resultant mixture is phase-separated by quenching at low temperature, resulting in crystallization of the first solvent while minimizing liquid-liquid demixing of the polymer solution. A leaching process is then performed to remove the macropore spacers. In certain embodiments, micropores can be created in the material by crystallization upon phase separation of the first solvent (i.e., the solvent in which the base polymer is soluble) and macropores are created by leaching which dissolves the macropore spacers. In some embodiments, the second solvent may be used to implement the leaching process.

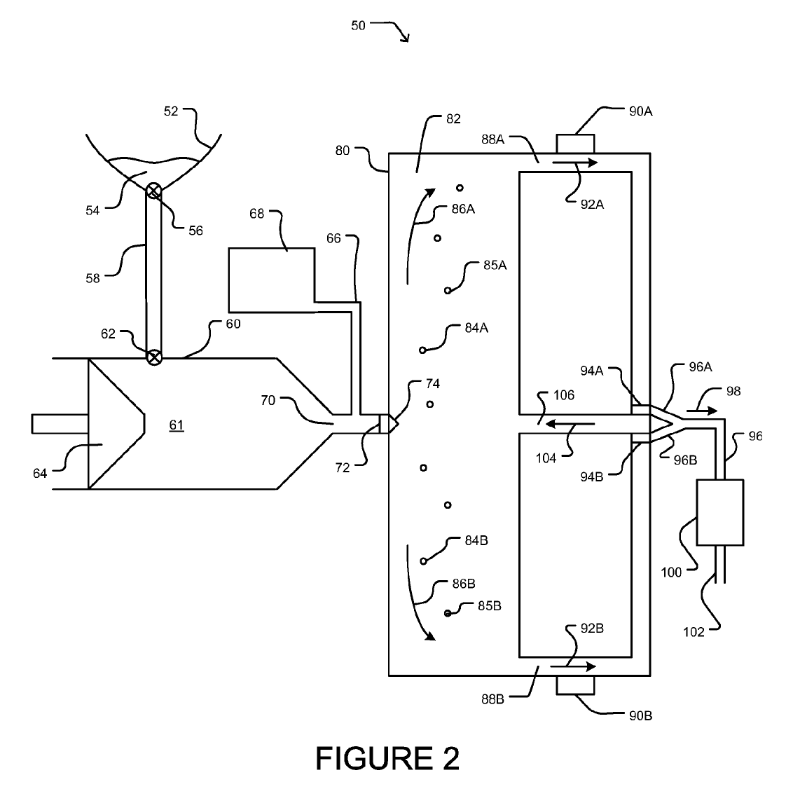

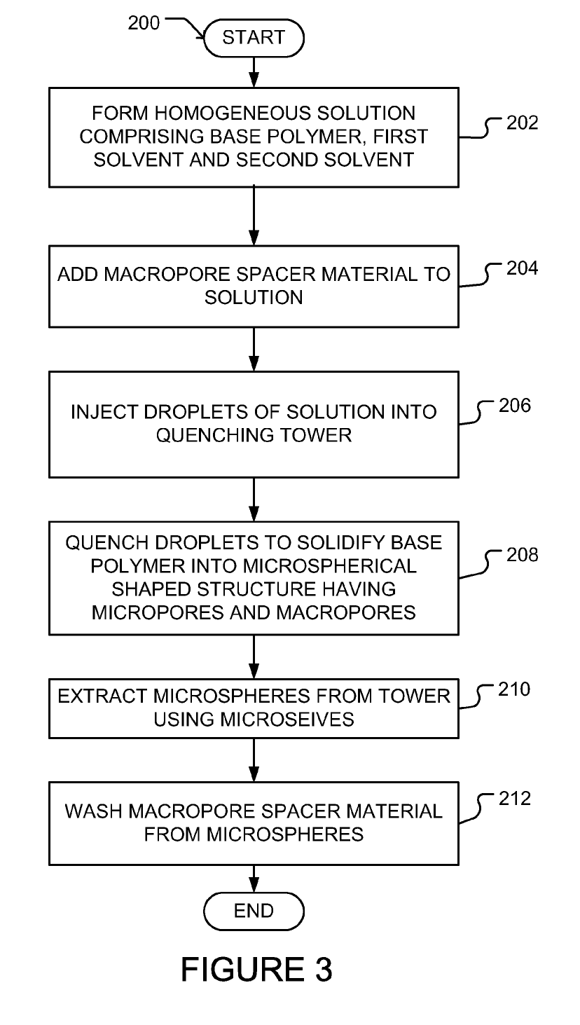

FIG. 2 illustrates an apparatus (50) which may be used to fabricate microspheres 20 according to a particular embodiment provided herein. FIG. 3 illustrates a block diagram of a particular embodiment of a method for fabricating microspheres 20 using apparatus 50.

By illustration only, method 200 begins in block 202, which involves preparing a homogeneous solution 54 by mixing base polymer 22 with a first solvent (in which the base polymer is soluble) and a second solvent in which the base polymer is insoluble, but which is miscible in the first solvent. In certain embodiments, the first and second solvents are miscible, and can form a mixture in which the base polymer is soluble. Without being bound by any theory, the selection of solvents and processing conditions are critical for solvent crystallization to occur before liquid-liquid demixing. The ratio of the amounts of first and second solvents in solution 54 may be selected to permit the base polymer to be substantially fully dissolved and to permit solution 54 to be substantially homogeneous. In certain embodiments, the volume ratio of the first solvent to the total volume of solvent, is between about 1% to about 50% v/v, about 1% to about 40% v/v, about 2% to about 30% v/v or about 4% to about 25% v/v. In a specific embodiment, the volume ratio of the first solvent to the total volume of solvent is about 5% to about 15% v/v.

The polymer concentration in the solvent mixture is between about 0.1% to about 50% by weight, between about 1% to about 40%, or about 5% to about 23% by weight. In a specific embodiment, the polymer concentration in the solvent mixture is between about 10% to about 20% by weight. The base polymer(s), the first and second solvents and macropore spacer material (68) can be selected from a variety of suitable materials.

In the illustrated apparatus 50, solution 54 is provided in a storage vessel 52. The vessel 52 can comprise an agitator, a mixer or the like (not shown) to ensure that the base polymer 22 is substantially fully dissolved in solution 54. In some embodiments, where macropore spacer particles 68 are soluble in the second solvent, then macropore spacer particles 68 may be combined with the solvent solution 54 in vessel 52. In other embodiments, macropore spacer particles 68 may be separately added via conduit 66 as described below. In the illustrated apparatus 50, vessel 52 is in fluid communication with injector 60 via conduit 58 which may comprise valves 56 and/or 62. In other embodiments, other suitable devices may be used to provide solution 54 to injector 60. In the illustrated embodiment, injector 60 comprises a chamber 61 into which solution 54 is provided and a pressure providing piston 64. In certain embodiments, piston 64 may be actuated by any suitable means to reduce the volume of chamber 61 and to thereby apply pressure to fluids contained therein as such fluids are directed toward outlet conduit 70 and nozzle 72.

By illustration only, method 200 then proceeds to block 204, which involves adding macropore spacer particles 68 to injector 60 via conduit 66. In certain embodiments, macropore spacers 68 may be carried in a small amount of one of the first or second solvents to facilitate addition of spacers 68 to solution 54. For example, macropore spacers 68 may be admixed with the first solvent to allow it to be more easily injectable into solution 54. In the illustrated embodiment, macropore spacers 68 are added to solution 54 in outlet conduit 70 (i.e. as close as reasonably possible to nozzle 72) and as temporally close as possible to injection of solution 54 into tower 80 via nozzle 72. Without being bound by any theory, this proximity of the addition of macropore spacers 68 to nozzle 72 tends to minimize separation between macropore spacers 68 and solution 54. In some embodiments, macropore spacers 68 is added in such a manner that they become reasonably evenly dispersed within solution 54 in outlet conduit 70. In other embodiments, other suitable mechanisms may be used to add macropore spacers 68 to solution 54. Such other suitable mechanisms may involve adding macropore spacers 68 to solution 54 in injector 60 or in other locations (e.g., in vessel 52). In some embodiments, apparatus 50 may comprise a mechanism or device for controlling the size of injected macropore spacer particles 68, such as by employing suitable sieves in conduit 66 or the like.

By illustration only, method 200 then proceeds to block 206, which involves injecting the mixture of solution 54 and macropore spacers 68 from injector 60 into quenching tower 80 in a manner which creates droplets 84A, 84B (collectively, droplets 84) of the mixture of solution 54 and macropore spacers 68 in tower 80. In the illustrated apparatus, droplets 84 are formed and injected from injector 60 into quenching tower 80 using a nozzle 72. In certain embodiments, droplets 84 are formed and injected from injector 60 into quenching tower 80 using a device that creates a fixed rate and aperature of laminar or nonlaminar dispersion into part 90. Nozzle 72 may comprise a one or more variable sized apertures 74 which may be varied to control the size of droplets 84. In some embodiments, the size of droplets 84 created by nozzle 72 is on the order of about 5 to about 1100 microns in diameter. Suitable nozzles or similar devices are known to those skilled in the art. In other embodiments, droplets 84 may be created using other suitable configured mistifying devices located between injector 60 and quenching tower 80. In certain embodiments, the rate of injection of droplets 84 through nozzle 72 and into tower 80 may be controlled by adjusting nozzle 72 and/or by adjusting the pressure applied to piston 64. In some embodiments, the rate of injection of droplets 84 into tower 80 should be controlled to facilitate quenching of droplets 84 as described herein.

By illustration only, method 200 then proceeds to block 208, which involves rapidly freezing (i.e. quenching) droplets 84 as soon as possible after droplets 84 enter tower 80. In certain embodiments, quenching tower 80 may comprise or otherwise be provided with means (not explicitly shown) for controlling or otherwise regulating the temperature and pressure therein. Such pressure and temperature regulation means are well known in the art and may include any suitable devices. In certain embodiments, the temperature and pressure within quenching tower 80 are regulated to induce quenching of droplets 84 as soon as possible after droplets 84 enter tower 80, as explained in more detail below.

In certain embodiments, quenching tower 80 is capable of supporting suitable pressures and temperatures for inducing rapid polymerization of base polymer 22 through phase change of the solvents from liquid to solid, before any significant dissociation of the liquids (e.g., liquid-liquid demixing of the first and second solvents) occurs in solution 54. In some embodiments, tower 80 may be filled with a suitable non-reactive medium 82 for effecting a rapid temperature drop. For example, medium 82 of tower 80 may comprise liquid nitrogen or other suitable coolants. In some embodiments, medium 82 of tower 80 may consist of or comprise the second solvent. In certain embodiments, the quenching of droplets 84 triggers crystallization of the first solvent and results in base polymer 22 solidifying out of solution (polymerizing). In some embodiments, base polymer 22 solidifies around macropore spacer particles 68, forming impressions within solidified base polymer 22 that correspond to macropores 24.

In certain embodiments, the phase change from liquid to solid also results in the formation of a network of micropores 26 within microsphere 20. Without wishing to be bound by any particular theory, it is believed that the quenching in tower 80 causes the crystallization of the first solvent in the solution, which in turn triggers the polymerization of base polymer 22 and the formation of micropores 26 in the resulting microsphere 20. Without being bound by any theory, it is believed that the second solvent (which is immiscible with base polymer 22 but miscible with the first solvent) acts as a nucleating agent that initiates the crystallization of the first solvent.

Because of the surface tension of the liquids in solution 54, in certain embodiments, droplets 84 (which are initially suspended in medium 82 of tower 80) are generally spherical or globular shaped (subject to deformation forces, such as gravity, forces which may be applied by injector 60 and the like). The result of generally spherical droplets 84 is that when droplets 84 are quenched in tower 80, the resultant solidified base polymer 22 retains the generally spherical shape which (as explained further below) provides generally microspherically shaped structures 85A, 85B (collectively, microspheres 85). In addition, the relatively small size of droplets 84 provides a large surface area to volume ratio, which improves quenching speed in relation to prior art processes which involve quenching in dish-shaped molds. While not wishing to be bound by any theory, faster quenching speed minimizes liquid-liquid separation during cooling. Liquid-liquid demixing can significantly reduce the formation of micropores 26 and thereby impact the bimodal pore distribution.

In the illustrated apparatus 50, tower 80 is vertically oriented, though in other embodiments tower 80 may be oriented in other directions. The mass density of droplets 84 and/or microspheres 85 may be greater than or less than the mass density of medium 82 in which they are suspended. In the illustrated embodiment, droplets 84A and/or microspheres 85A that are less dense than medium 82 will tend to rise in direction 86A. In contrast, droplets 84B and/or microspheres 85B that are more dense than medium 82 will tend to sink in direction 86B.

By illustration only, method 200 then proceeds to block 210, which involves extracting microspheres 85 from tower 80. In the illustrated embodiment, the block 210 extraction is performed using microsieves 94A, 94B (collectively, microsieves 94). More particularly, microspheres 85 may be extracted from tower 80 by way of one or both of outlet channels 88A, 88B (collectively outlet channels 88) which respectively incorporate microsieves 94A, 94B. One or more suitably configured pumps 90A, 90B (collectively pumps 90) may be used to circulate medium 82 carrying microspheres 85 around a loop from tower 80, through output channels 88, through microsieves 94, and back into tower 80 through a return channel 106. Part 92A is a narrow conduit, for example, tubing or other flexible substance that would allow for a continuous circuit of fluid flow to promote the movements of the microspheres into the sieve. In certain embodiments, part 92A is tubing that would be attached to part 90A, which would be, for example, a roller pump, thermal pump or other mechanism to promote a flow of fluid through the circuit. In the illustrated embodiment, upper output channel 88A is connected at or near the top of tower 80 to extract microspheres 85A that rise within medium 82 and lower output channel 88B is connected at or near the bottom of tower 80 to extract microspheres 85B that sink within medium 82.

It will be appreciated that apparatus 50 may comprise any suitable mechanism for circulating medium 82 through microsieves 94 and that embodiments of apparatus 50 are not limited to the particular circulation paths and pumping arrangements shown in FIG. 3. In certain embodiments, the use of a pair of output channels 88 is not necessary. In some embodiments, microspheres 85 may be forced through a single output channel and a corresponding microsieve by a suitably configured pump. In some embodiments, one of upper output channel 88A or lower output channel 88B may be operational and the other one of channels 88 may be shut off (e.g., by a suitably configured valve). The one of channels 88 that may be selected for operation may depend on the expected mass density of microspheres 85. In still other embodiments, apparatus 50 may comprises a different number of output channels 88. Pumps 90 may comprise any suitable type of pump, such as a roller pump, for example, and may be used in any suitable pumping arrangement. One or more pumps 90 may be employed depending on the circulation paths that are most suitable for the density of microspheres 85 being fabricated.

Microsieves 94 may be designed to capture microspheres 85 while allowing underlying medium 82 to pass therethrough. Microspheres 85 captured in microsieves 94 may then be extracted (e.g., manually or by any other suitable means) for washing and further processing.

By illustration only, method 200 then proceeds to block 212, which involves washing off any residual macropore spacers 68 and solvents from microspheres 85, resulting in clean microspheres 20 comprising only base polymer 22 with bimodal pore distributions. The block 212 washing process may not be necessary if microspheres 85 are sufficient cleaned as they circulate through medium 82 in tower 80 (depending on the choice of medium 82). The block 212 washing process for removing macropore spacers 68 may involve leaching (i.e. dissolution of macropore spacers 68 in a suitable liquid or gas solvent), application of heat and/or sublimation or other suitable techniques for removal of residual macropore spacers 68 and solvents. For example, if macropore spacers 68 are salt crystals or other water-soluble materials, the washing process may involve leaching microspheres 85 in water. As another example, microspheres 85 may be placed in a vessel connected to a vacuum pump for a time needed for complete sublimation of the solvents. In some embodiments, the block 212 washing process need not remove macropore spacers 68. For example, macropore spacers 68 may comprise bioactive substances that may initially remain incorporated into the macropore of microspheres 20 as opposed to being washed out and which may subsequently be taken up by the organism into which microspheres 20 are deployed. At the conclusion of block 212, microspheres 20 are ready for further processing and/or application as described further below.

In some embodiments, method 200 may comprise an additional step (not explicitly shown) of sterilizing microspheres 20. By way of non-limiting example, such sterilization may involve using suitable external radiation or gas-based sterilization processes. In some embodiments, method 200 may also involve sorting microspheres 20 by size (not explicitly shown) so that microspheres 20 within a particular size range may be selected for particular applications. Such sorting may be accomplished, for example, using an arrangement of microsieves of varying fineness.

### 1. Base Polymer

In certain embodiments, base polymer 22 comprises a biocompatible polymer or monomer, which can also be bioabsorbable and/or biodegradable. In some embodiments, the base polymer is sufficiently mechanically rigid at room temperatures and body temperatures, such that the microspheres maintain their shape and pore structure during the washing phase of the fabrication process and during subsequent processing and application in vivo.

Non-limiting examples of bioabsorbable polymers that are suitable for use as a base polymer (or at least a part of a base polymer) include one or more of:

(i) microorganism-based polymers:
(a) polyhydroxy-alkanoates (PHA)—e.g., poly(hydroxybutyrate) (PHB), poly(hydroxybutyrate co-hydroxyvaerate) (PVBV);

(ii) biotechnology derived monomers:
(a) alpha-hydroxycarboxilic and copolymers thereof—e.g., poly(lactic) acids (PLA), poly(gloycolide) (PGA);

(iii) petrochemical-based polymers:
(a) polycaprolactones (PCL);
(b) polyesteramids;
(c) alphatic co-polyesters;
(d) aromatic co-polyesters.

Other non-limiting examples of bioabsorbable materials that could be used to provide a base polymer (or at least a part of a base polymer) include absorbable biocompatible biomass products such as one or more of:

(i) polysaccharides:
(a) starches—e.g., wheat, corn and/or potato;
(b) ligno-cellulosic materials—e.g., wood, straw;
(c) other polysaccharide materials—e.g., pectin, chotosan/chitin, gums, waxes;

(ii) proteins and lipids:
(a) animal-based proteins and lipids—e.g. casein, whey, collagen/gelatin, fibrin, glycosaminoglycans (GAGS);
(b) plant-based proteins and lipids—e.g. zein, soya, gluten.

Additional non-limiting examples suitable for a base polymers as used herein include one or more of: polyethylene oxide/polyethylene terephthalate and copolymers thereof; copolymers of lactic or glycolic acid or combinations of the two with hydroxy-ended flexible chains, such as poly(alkylene glycols) of various molecular weights and forms and commercially available. Examples of poly(alkylene glycols) include, but are not limited to, hydroxyl-terminated polyethylene oxide, polypropylene oxide, poly(oxyethylene-co-oxypropylene) and polytetramethylene oxide chains, poly(oxyethylene glycols), poly(oxypropylene)-poly(oxyethylene)-glycols block copolymers and poly(oxybutylene)glycols.

Further non-limiting examples suitable for a base polymer as used herein include one or more of: biodegradable and biocompatible polycaprolactones, polyhydroxybutyrates and copolymers of polyesters, polycarbonates, polyanhydrides and poly(ortho esters); bisphenol-A based polyphosphoesters such as poly(bisphenol-A phenylphosphate), poly(bisphenol-A ethylphosphate), poly(bisphenol-A ethylphosphonate), poly(bisphenol-A phenylphosphonate), poly[bis(2-ethoxy)hydrophosphonic terephthalate], and copolymers of bisphenol-A based poly(phosphoesters); polymers derived from tyrosine-derived diphenol monomers having an exemplary structure as follows:

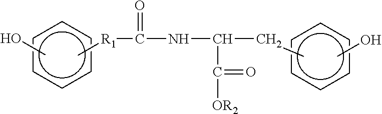

Wherein R₁ is —CH═CH— or (—CH₂—)ₙ, in which n is zero or an integer from one to eight; and R₂ is selected from straight and branched alkyl and alkylaryl groups containing up to 18 carbon atoms. The diphenol compounds can be polymerized to form, for example, polyiminocarbonates, polycarbonates, polyacrylates, polyurethanes or polyethers. See, e.g., U.S. Pat. Nos. 5,099,060 and 5,198,507 for methods of preparing polyiminocarbonates and polycarbonates. Suitable diphenol monomers for use in the methods provided herein include, by way of illustration, desaminotyrosyl-tyrosine (DT) esters such as desaminotyrosyl tyrosine ethyl ester (DTE), desaminotyrosyl tyrosine butyl ester (DTB), desaminotyrosyl tyrosine hexyl ester (DTH), desaminotyrosyl tyrosine octyl ester (DTO), or a combination thereof.

Still further non-limiting examples suitable for a base polymer includes one or more of: polycarbonates, polyimino-carbonates, polyarylates, polyurethanes, strictly alternating poly(alkylene oxide ethers), poly(alkylene oxide) block copolymers polymerized from dihydroxy monomers prepared from α- and β-hydroxy acids and derivatives of tyrosine, block copolymers of polycarbonates and polyarylates with poly(alkylene oxides), polycarbonates, polyimino carbonates, polyarylates, poly(alkylene oxide) block copolymers. block copolymers of polycarbonates with poly(alkylene oxides), block copolymers of polyarylates with poly(alkylene oxides), α-hydroxycarboxylic acids, poly(capro-lactones), poly(hydroxybutyrates), polyanhydrides, poly(ortho esters) and polyesters bisphenol-A based poly(phosphoesters), or a combination thereof. See, e.g., U.S. Pat. No. 5,216,115 for methods of preparing polyarylates, which is incorporated herein by reference in their entirety.

In one embodiment the particle (or microsphere) comprises polyvinyl alcohol. In another embodiment, the particle comprises an acrylic, acrylamide or acrylate polymer or copolymer. In some embodiments, the particle comprises a polyvinyl alcohol and an acrylic, acrylamide or acrylate polymer or copolymer. In one embodiment, the particle comprises a trisacrylamide polymer. In another embodiments, the particle the base polymer is N-tris-hydroxymethyl methylacrylamide, diethylaminoathylacrylamide, N,N-methylene-bis-acrylamide, or a combination thereof. In one embodiments, the particle comprises a sodium acrylate and vinyl alcohol copolymer. In certain embodiments, the particle further comprises a cell adhesion promoter, such as a gelatin. In some embodiments, the particle is crosslinked. In other embodiments, the particle is not crosslinked.

The above-described materials are non-limiting examples of possible materials for use as (or as part of) a base polymer. Still other non-limiting examples of such materials include iodine impregnated polymers or polymers containing free carboxylic acid pendent chains. It may also be possible to use apatite derivatives, such as hydroxyapatite and flourapatite, for a base polymer. Any of the above-described materials, or materials provided elsewhere herein, may be used alone or in any combination, for example as a monomer, polymer or copolymer thereof.

### 2. Solvents

In certain embodiments, the first solvent may be characterized by being a solvent in which a base polymer 22 is soluble in varying concentrations. In some embodiments, the first solvent is also miscible in the second solvent to form a continuous phase medium. In particular embodiments, the first solvent may have a melting point between about −20° C. and about +20° C., such that, at a high rate of cooling, crystallization is the favored phase separation mechanism (though the first solvent is not limited to this temperature range) and, in other particular embodiments, solvents may have melting points between about −40° C. and about +40° C. An example of a compatible first solvent for a polylactic acid (PLA) derived base polymer is 1,4-dioxane, which has a melting point of 12° C. and a low crystallization energy. Other solvents may be used for other base polymer materials.

In certain embodiments, the second solvent may be characterized by being a solvent in which a base polymer is immiscible or only miscible in very low concentrations. The second solvent is, however, completely miscible in the first solvent. A specific example of a second solvent compatible with a PLA base polymer and a 1,4-dioxane first solvent is water. Other non-limiting examples of solvents that are suitable for use as the second solvent include alcohols such as, but not limited to, methanol, ethanol, isopropanol, tert-butanol and 1,3-propanediol. Alternatively, a second solvent may also serve as a continuous phase in an emulsion consisting of a first solvent, a macropore spacer and an optional additive.

### 3. Macropore Spacer Material

Macropore spacer material 68 may be characterized, in some embodiments, by being immiscible or only slightly miscible in the first solvent and base polymer 22. In some embodiments, the macropore spacer material is miscible in the second solvent. In one embodiment, the macropore spacer material is largely miscible in the second solvent. For example, sodium chloride (table salt) provides a suitable macropore spacer material in the case where the second solvent is water. Salt has the additional benefit of being biocompatible should any residual quantities remain in microspheres 20 after washing (block 212). In some embodiments, macropore spacer material 68 may not be miscible in either the first or second solvents. In certain embodiments, macropore spacer material 68 may be soluble in a third solvent used in the washing process 212 described above. In some embodiments, macropore spacer material 68 may be miscible in medium 82 of tower 80, such that it is washed out during circulation through tower 80. In some embodiments, macropore spacer material 68 may intentionally not be washed out such that it remains (at least initially) an additive incorporated into microsphere 20 for later application (for example, cisplatin may be used as macropore spacer material 68 where medium 82 is nitrogen based). Macropore spacer 68 may also be non-absorbable, dislodging from microsphere 20 as a result of bioerosion and resulting in embolization of smaller luminal structures (such as blood vessels).

As will be appreciated, control of the size of macropores 24 may depend on the overall size of crystals or particles used to provide macropore spacers 68, as well as the timing of the introduction of macropore spacers 68 into solution 54. In addition to or as an alternative to salt(s), other non-toxic biocompatible crystalline substance satisfying any of the solubility criteria discussed above may also be suitable as a macropore spacer material 68. By way of non-limiting example, suitable macropore spacer materials 68 may include: biologically acceptable alkali metal and alkaline earth metal halides, phosphates, sulfates, and the like; crystals of sugars; microspheres of water-soluble polymers; and proteins, such as albumin. In a specific embodiment, the macropore spacer material 68 as used herein is sodium chloride. Macropore spacer material 68 may also include smaller microspheres 20 or nanoparticles (which may or may not be bioabsorbable. Particles of these materials should be selected having the diameter that is desired for macropores 24.

In certain embodiments, the macropore spacer material is an additive. In some embodiments, the macropore spacer material is cisplatin. In one embodiment, the macropore spacer material is cisplatin and the medium is a nitrogen-based medium. In certain embodiments, the macropore spacer material is bioabsorbable. In other embodiments, the macropore spacer material is not bioabsorbable. In certain embodiments, the macropore spacer material embolizes luminal structures, such as blood vessels. In certain embodiments, wherein the macropore spacer material is non-toxic and/or biocompatible. In some embodiments, the macropore spacer material is selected from the group consisting of an alkali metal and alkaline earth metal halides, phosphates, sulfates; sugars, crystals of sugars; water soluble polymers, microspheres, nanoparticles, microspheres of water-soluble polymers; proteins, albumin, and sodium chloride.

### 4. Additives

In some embodiments, methods of fabricating microspheres 20 do not comprise one or more additives (e.g., bio-active material) into microspheres 20. In other embodiments, methods of fabricating microspheres 20 may further comprise incorporating one or more additives (e.g., bio-active material) into microspheres 20. This step may be performed at various stages of the fabrication process. By way of non-limiting example, an additive could be introduced to polymer solution 54 before its injection into tower 80, added to the pores of microspheres 20 after washing (block 212) and/or incorporated into macropore spacers 68 which initially remain present in microspheres 20, or additives may act as initiators to the polymerization process.

In certain embodiments, an additive capable of withstanding the temperature fluctuations in the fabrication process is incorporated into polymer solution 54 before injection into tower 80. In such embodiments, the additive can be provided to the solution within about 1, 5, 10, 15, 20, 30, 45 minutes or about 1, 2, 4, 6 hours of injection. In some embodiments, base polymer 22 and the first and second solvents may be pre-blended before the additive is dissolved therein or the additive may be dissolved in the solvent in which it is most soluble, after which the first and second solvents and base polymer 22 may be combined. Such additive materials can become embedded in base polymer 22 when droplets 84 are quenched in tower 80. During subsequent stages of formation of microspheres 20 (e.g., after quenching of droplets 84 to form microspheres 85), such additives may adhere to the surface of microspheres 20 by a variety of means such as through cross linking with base polymer 22, ionic bonding, acid base reactions, receptor site attraction or gravitational forces. In certain embodiments, the additive is provided to the solution after injection. In such embodiments, the additive is provided to the solution within about 1, 5, 10, 15, 20, 30, 45 minutes or about 1, 2, 4, 6 hours after injection. In some embodiments, the additive is provided to the solution during injection.

In certain embodiments, the additive is provided to the solution prior to quenching. In such embodiments, the additive can be provided to the solution within about 1, 5, 10, 15, 20, 30, 45 minutes or about 1, 2, 4, 6 hours of quenching. In other embodiments, the additive is provided to the solution after quenching. In such embodiments, the additive is provided to the solution within about 1, 5, 10, 15, 20, 30, 45 minutes or about 1, 2, 4, 6 hours after quenching. In some embodiments, the additive is provided to the solution during quenching.

In certain embodiments, the additive is provided to the solution prior to washing. In such embodiments, the additive can be provided to the solution within about 1, 5, 10, 15, 20, 30, 45 minutes or about 1, 2, 4, 6 hours of washing. In other embodiments, the additive is provided to the solution after washing. In such embodiments, the additive is provided to the solution within about 1, 5, 10, 15, 20, 30, 45 minutes or about 1, 2, 4, 6 hours after washing. In some embodiments, the additive is provided to the solution during washing.

Additionally or alternatively to introduction of additives to microspheres 20 during their fabrication, additives may be incorporated into or coated on microspheres 20 after the conclusion of method 200. In certain embodiments, the additive can be covalently attached to the polymer. In one embodiment, the additive is covalently attached to polymers having pendent free carboxylic acid groups using methods known in the art. See, for example, U.S. Pat. Nos. 5,219,564 and 5,660,822; Nathan et al., Bio. Congo Chem., 4, 54-62 (1992) and Nathan, Macromolecules, 25, 4476 (1992), which are incorporated herein by reference in their entirety. In certain embodiments, hydrolytically stable conjugates are utilized when the additive is active in conjugated form. Hydrolyzable conjugates are utilized when the additive is inactive in conjugated form.

In certain embodiments, the additive can be incorporated into or coated onto the particle. In one embodiment, microspheres 20 can be coated with an anticoagulant (such as heparin) such that the anticoagulant becomes covalently attached to the surface of microsphere 20 through processes known in the art. Such a non-thrombogenic coating may be beneficial in applications such as tissue engineering or stem cell transplantation or harvesting. In some embodiments, microspheres 20 may be coated with bioactive substances that function as receptors or chemoattractors for a desired population of cells. Such coatings may be applied through absorption or chemical bonding. In certain embodiments, the additive adheres to a surface of the particle. In one embodiment, the additive adheres to the surface of the microsphere by cross-linking with the base polymer, ionic bonding, acid base reactions, receptor site attraction or gravitational forces.

In certain embodiments, additives may be subsequently released from microsphere 20 in a controlled fashion in a selected target area of a living subject or may be activated by contact with surrounding fluids and tissues without being released from microsphere 20. Additives may be released by a bioerosion of microspheres 20 (if base polymer 22 is biodegradable), by diffusion from microspheres 20, or by migration to the polymer surface of microspheres 20. The additive may be provided in a physiologically or pharmaceutical acceptable carrier, excipient, stabilizer, etc., and may be provided in sustained release or timed release formulations. The additives may also incorporate agents to facilitate their delivery, such as antibodies, antibody fragments, growth factors, hormones, or other targeting moieties, to which the additives are coupled.

Additives suitable for use with microspheres 20 include biologically or pharmaceutically active compounds. Examples of biologically active compounds include, but are not limited to, cell attachment mediators, such as the peptide containing variations of the “RGD” integrin binding sequence known to affect cellular attachment, biologically active ligands, and substances that enhance or exclude particular varieties of cellular or tissue ingrowth. Such substances include, for example, osteoinductive substances, such as bone morphogenic proteins (BMP), epidermal growth factor (EGF), fibroblast growth factor (FGF), platelet-derived growth factor (PDGF), insulin-like growth factor (IGF-I and II), TGF-β, vascular endothelial growth factor (VEGF), tumor necrosis factor (TNF), tumor induction factor (TIF), and the like.

Examples of pharmaceutically active compounds include, but are not limited to, acyclovir, cephradine, malfalen, procaine, ephedrine, adriomycin, daunomycin, plumbagin, atropine, guanine, digoxin, quinidine, biologically active peptides, chlorin e 6, cephalothin, proline and proline analogues such as cis-hydroxy-L-proline, penicillin V, aspirin, ibuprofen, steroids, nicotinic acid, chemodeoxycholic acid, chlorambucil, and the like. Other non-limiting example bioactive substances that may be used as additives include vasodilators such as nitrates (nitroglycerin, nitrous oxide based materials), calcium channel blockers (commercial name verapamil), thromobolyics (such as tissue plasminogen factor (TPA), Streptokinase, Urokinase), antiplatelet aggregation/adhesion factors (such as IIa-IIIb inhibitors, commercial name reopro).

Other potential additives include conventional or biological chemotherapeutics. Non-limiting examples of conventional chemotherapeutics include platinum class chemotherapeutics, topoisomers, topo inhibitors and reverse transcriptase chemotherapeutics. Non-limiting examples of biological chemotherapeutics include bevacizumab, cetuximab, 3-bromopyruvate, small molecule biologics such as sorafenib and multikinase inhibitors. Other exemplary additives are the therapeutic agents and drugs provided elsewhere herein.

In certain embodiments, the additive as used in conjunction with the bimodal particles or microspheres, compositions and methods provided herein is one or more therapeutic agents. Therapeutic agents that can be used in combination with the microspheres in the compositions, methods or kits provided herein include (e.g., one, two, three, four or more) agent(s), such as a drug. Such a therapeutic agent can be any one or more of an anti-neoplastic drug, anti-angiogenesis drug, anti-fungal drug, anti-viral drug, anti-inflammatory drug, anti-bacterial drug, a cytotoxic drug, a chemotherapeutic or pain relieving drug and/or an anti-histamine drug. The therapeutic agent can also be, for example, any one or more of hormones, steroids, vitamins, cytokines, chemokines, growth factors, interleukins, enzymes, anti-allergenic agents, circulatory drugs, anti-tubercular agents, anti-anginal agents, anti-protozoan agents, anti-rheumatic agents, narcotics, cardiac glycoside agents, sedatives, local anesthetic agents, general anesthetic agents, and combinations thereof. Such therapeutic agents can also include, for example, antineoplastic, angiogenic factors, immuno-suppressants, or antiproliferatives (anti-restenosis agents). Other non-limiting examples of therapeutic agents include embryonic factors, fibroblast growth factors, transcription factors, kinase inhibitors, or adenosine. In certain embodiments, the therapeutic agent is an anti-neoplastic, chemotherapeutic or pain relieving drug.

Examples of anti-angiogenic or anti-neoplastic drugs include, but are not limited to, AGM-1470 (TNP-470), angiostatic steroids, angiostatin, antibodies against avβ3, antibodies against bFGF, antibodies against IL-1, antibodies against TNF-α, antibodies against VEGF, auranofin, azathioprine, BB-94 and BB-2516, basic FGF-soluble receptor, carboxyamido-trizole (CAI), cartilage-derived inhibitor (CDI), chitin, chloroquine, CM 101, cortisone/heparin, cortisone/hyaluroflan, cortexolone/heparin, CT-2584, cyclophosphamide, cyclosporin A, dexamethasone, diclofenac/hyaluronan, eosinophilic major basic protein, fibronectin peptides, Glioma-derived angiogenesis inhibitory factor (GD-AIF), GM 1474, gold chloride, gold thiomalate, heparinases, hyaluronan (high and low molecular-weight species), hydrocortisonelbeta-cyclodextran, ibuprofen, indomethacin, interferon-α, interferon γ-inducible protein 10, interferon-γ, IL-1, IL-2, IL-4, IL-12, laminin, levamisole, linomide, LM609, martmastat (BB-2516), medroxyprogesterone, methotrexate, minocycline, nitric oxide, octreotide (somatostatin analogue), D-penicillamine, pentosan polysulfate, placental proliferin-related protein, placental RNase inhibitor, plasminogen activator inhibitor (PAIs), platelet factor-4 (PF4), prednisolone, prolactin (16-kDa fragment), proliferin-related protein, prostaglandin synthase inhibitor, protamine, retinoids, somatostatin, substance P, suramin, SU101, tecogalan sodium (05-4152), tetrahydrocortisol-sthrombospondins (TSPs), tissue inhibitor of metalloproteinases (TIMP 1, 2, 3), thalidomide, 3-aminothalidomide, 3-hydroxythalidomide, metabolites or hydrolysis products of thalidomide, 3-aminothalidomide, 3-hydroxythalidomide, vitamin A and vitreous fluids. In another embodiment, the anti-angiogenic agent is selected from the group consisting of thalidomide, 3-aminothalidomide, 3-hydroxythalidomide and metabolites or hydrolysis products of thalidomide, 3-aminothalidomide, 3-hydroxythalidomide. In one embodiment, the anti-angiogenic agent is thalidomide.

Other anti-angiogenic or anti-neoplastic drugs include, without limitation, alkylating agents, nitrogen mustards, antimetabolites, gonadotropin releasing hormone antagonists, androgens, antiandrogens, antiestrogens, estrogens, and combinations thereof. Specific examples include but are not limited to actinomycin D, aldesleukin, alemtuzumab, alitretinoin, allopurinol, altretamine, amifostine, aminoglutehimide, amphotercin B, amsacrine, anastrozole, ansamitocin, arabinosyl adenine, arsenic trioxide, asparaginase, aspariginase Erwinia, BCG Live, benzamide, bevacizumab, bexarotene, bleomycin, 3-bromopyruvate, busulfan, calusterone, capecitabine, carboplatin, carzelesin, carmustine, celecoxib, chlorambucil, cisplatin, cladribine, cyclophosphamide, cytarabine, cytosine arabinoside, dacarbazine, dactinomycin, darbepoetin alfa, daunorubicin, daunomycin, denileukin diftitox, dexrazoxane, dexamethosone, docetaxel, doxorubicin, dromostanolone, epirubicin, epoetin alfa, estramustine, estramustine, etoposide, VP-16, exemestane, filgrastim, floxuridine, fludarabine, fluorouracil (5-FU), flutamide, fulvestrant, demcitabine, gemcitabine, gemtuzumab, goserelin acetate, hydroxyurea, ibritumomab, idarubicin, ifosfamide, imatinib, interferon (e.g., interferon α-2a, interferon α-2b), irinotecan, letrozole, leucovorin, leuprolide, lomustine, meciorthamine, megestrol, melphalan (e.g., PAM, L-PAM or phenylalanine mustard), mercaptopurine, mercaptopolylysine, mesna, mesylate, methotrexate, methoxsalen, mithramycin, mitomycin, mitotane, mitoxantrone, nandrolone phenpropionate, nolvadex, oprelvekin, oxaliplatin, paclitaxel, pamidronate sodium, pegademase, pegaspargase, pegfilgrastim, pentostatin, pipobroman, plicamycin, porfimer sodium, procarbazine, quinacrine, raltitrexed, rasburicase, riboside, rituximab, sargramostim, spiroplatin, streptozocin, tamoxifen, tegafur-uracil, temozolomide, teniposide, testolactone, tioguanine, thiotepa, tissue plasminogen activator, topotecan, toremifene, tositumomab, trastuzumab, treosulfan, tretinoin, trilostane valrubicin, vinblastine, vincristine, vindesine, vinorelbine, zoledronate, salts thereof, or mixtures thereof. In some embodiments, the platinum compound is spiroplatin, cisplatin, or carboplatin. In specific embodiments, the drug is cisplatin, mitomycin, paclitaxel, tamoxifen, doxorubicin, tamoxifen, or mixtures thereof.

Examples of pain reliving drugs are, without limitation, analgesics or anti-inflammatories, such as non-steriodal anti-inflammatory drugs (NSAID), ibuprofen, ketoprofen, dexketoprofen, phenyltoloxamine, chlorpheniramine, furbiprofen, vioxx, celebrex, bexxstar, nabumetone, aspirin, codeine, codeine phosphate, acetaminophen, paracetamol, xylocalne, and naproxin. In some embodiments, the pain relieving drug is an opioid. Opioids are commonly prescribed because of their effective analgesic, or pain relieving, properties. Among the compounds that fall within this class include narcotics, such as morphine, codeine, and related medications. Other examples of opioids include oxycodone, propoxyphene, hydrocodone, hydromorphone, and meperidine. Narcotics, include, for example, without limitation, paregoric and opiates, such as codeine, heroin, methadone, morphine and opium.

Hormones and steroids, include, for example, without limitation, growth hormone, melanocyte stimulating hormone, adrenocortiotropic hormone, dexamethasone, dexamethasone acetate, dexamethasone sodium phosphate, cortisone, cortisone acetate, hydrocortisone, hydrocortisone acetate, hydrocortisone cypionate, hydrocortisone sodium phosphate, hydrocortisone sodium succinate, prednisone, prednisolone, prednisolone acetate, prednisolone sodium phosphate, prednisolone tebutate, prednisolone pivalate, triamcinolone, triamcinolone acetonide, triamcinolonehexacetonide, triamcinolone acetate, methylprednisolone, methylprednisolone acetate, methylprednisolone sodium succinate, flunsolide, beclomethasone dipropionate, betamethasone sodium phosphate, betamethasone, vetamethasone disodium phosphate, vetamethasone sodium phosphate, betamethasone acetate, betamethasone disodium phosphate, chloroprednisone acetate, corticosterone, desoxycorticosterone, desoxycorticosterone acetate, desoxycorticosterone pivalate, desoximethasone, estradiol, fludrocortisone, fludrocortisoneacetate, dichlorisone acetate, fluorohydrocortisone, fluorometholone, fluprednisolone, paramethasone, paramethasone acetate, androsterone, fluoxymesterone, aldosterone, methandrostenolone, methylandrostenediol, methyl testosterone, norethandrolone, testosterone, testosteroneenanthate, testosterone propionate, equilenin, equilin, estradiol benzoate, estradiol dipropionate, estriol, estrone, estrone benzoate, acetoxypregnenolone, anagestone acetate, chlormadinone acetate, fluorogestone acetate, hydroxymethylprogesterone, hydroxymethylprogesterone acetate, hydroxyprogesterone, hydroxyprogesterone acetate, hydroxyprogesterone caproate, melengestrol acetate, normethisterone, pregnenolone, progesterone, ethynyl estradiol, mestranol, dimethisterone, ethisterone, ethynodiol diacetate, norethindrone, norethindrone acetate, norethisterone, fluocinolone acetonide, flurandrenolone, flunisolide, hydrocortisone sodium succinate, methylprednisolone sodium succinate, prednisolone phosphate sodium, triamcinolone acetonide, hydroxydione sodium spironolactone, oxandrolone, oxymetholone, prometholone, testosterone cypionate, testosterone phenylacetate, estradiol cypionate, and norethynodrel.

Peptides and peptide analogs, include, for example, without limitation, manganese super oxide dismutase, tissue plasminogen activator (t-PA), glutathione, insulin, dopamine, peptide ligands containing RGD, AGD, RGE, KGD, KGE or KQAGDV (peptides with affinity for theGPEXma receptor), opiate peptides, enkephalins, endorphins and their analogs, human chorionicgonadotropin (HCG), corticotropin release factor (CRF), cholecystokinins and their analogs, bradykinins and their analogs and promoters and inhibitors, elastins, vasopressins, pepsins, glucagon, substance P, integrins, captopril, enalapril, lisinopril and other ACE inhibitors, adrenocorticotropic hormone (ACTH), oxytocin, calcitonins, IgG or fragments thereof, IgA or fragments thereof, IgM or fragments thereof, ligands for Effector Cell Protease Receptors (all subtypes), thrombin, streptokinase, urokinase, t-PA and all active fragments or analogs, Protein Kinase C and its binding ligands, interferons (α-IFN, β-IFN, γ-IFN), colony stimulating factors (CSF), granulocyte colony stimulating factors (GCSF), granulocyte-macrophage colony stimulating factors (GM-CSF), tumor necrosis factors (TNF), nerve growth factors (NGF), platelet derived growth factors, lymphotoxin, epidermal growth factors, fibroblast growth factors, vascular endothelial cell growth factors, erythropoietin, transforming growth factors, oncostatin M, interleukins (IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, etc.), metalloprotein kinase ligands, collagenases and agonists and antagonists.

Antibodies, include, for example, without limitation, substantially purified antibodies or fragments thereof, including non-human antibodies or fragments thereof. In various embodiments, the substantially purified antibodies or fragments thereof, can be human, non-human, chimeric and/or humanized antibodies. Such non-human antibodies can be goat, mouse, sheep, horse, chicken, rabbit, or rat antibodies. The antibodies can be monoclonal or polyclonal antibodies.

Anti-mitotic factors include, without limitation, estramustine and its phosphorylated derivative, estramustine-phosphate, doxorubicin, amphethinile, combretastatin A4, and colchicine.

Anti-coagulation agents, include, for example, without limitation, phenprocoumon and heparin.

Anti-viral agents, include, for example, without limitation, acyclovir, amantadine azidothymidine (AZT or Zidovudine), ribavirin, and vidarabine monohydrate (adenine arabinoside,ara-A).

Anti-anginal agents, include, for example, without limitation, diltiazem, nifedipine, verapamil, erythritol tetranitrate, isosorbide dinitrate, nitroglycerin (glyceryl trinitrate), and pentaerythritolteiranitrate.

Antibiotics, include, for example, dapsone, chloramphenicol, neomycin, cefaclor, cefadroxil, cephalexin, cephradine erythromycin, clindamycin, lincomycin, amoxicillin, ampicillin, bacampicillin, carbenicillin, dicloxacillin, cyclacillin, picloxacillin, hetacillin, methicillin, nafcillin, oxacillin, penicillin G, penicillin V, ticarcillin, rifampin, and tetracycline.

Anti-inflammatory agents and analgesics, include, for example, diflunisal, ibuprofen, indomethacin, meclofenamate, mefenamic acid, naproxen, oxyphenbutazone, phenylbutazone, piroxicam, sulindac, tolmetin, aspirin and salicylates.

Circulatory drugs, include, for example, without limitation, propranolol.

Cardiac glycoside agents, include, for example, without limitation, deslanoside, digitoxin, digoxin, digitalin and digitalis.

Neuromuscular blocking agents, include, for example, without limitation, atracurium mesylate, gallamine triethiodide, hexafluorenium bromide, metocurine iodide, pancuronium bromide, succinylcholine chloride(suxamethonium chloride), tubocurarine chloride, and vecuronium bromide.

Sedatives, include, for example, without limitation, amobarbital, amobarbital sodium, aprobarbital, butabarbital sodium, chloral hydrate, ethchlorvynol, ethinamate, flurazepam hydrochloride, glutethimide, methotrimeprazine hydrochloride, methyprylon, midazolam hydrochloride paraldehyde, pentobarbital, pentobarbital sodium, phenobarbital sodium, secobarbital sodium, talbutal, temazepam, and triazolam.

Local anesthetic agents, include, for example, without limitation, bupivacaine hydrochloride, chloroprocaine hydrochloride, etidocaine hydrochloride, lidocaine hydrochloride, mepivacaine hydrochloride, procaine hydrochloride, and tetracaine hydrochloride.

General anesthetic agents, include, for example, without limitation, droperidol, etomidate, fentanyl citrate with droperidol, ketamine hydrochloride, methohexital sodium, and thiopental sodium.

Radioactive particles or ions, include, for example, without limitation, strontium, rhenium, yttrium, technetium, and cobalt.

In other applications, microspheres 20 may provide a delivery vehicle for various therapies or other additives to localized target areas in the body, allowing for extended, controlled exposure of the therapy or other additive to the target area. Microspheres 20 may be doped, admixed, coated, or impregnated with a desired therapeutic agent or other additive and injected into to the targeted area by any suitable means. For example, microspheres 20 could potentially deliver any of the following additives: chemotherapy, immunomodulators, viral vectors, chemoattractants, polypeptides, neurotransmitters, biologics (e.g. bevacizumab), antibody receptor sites, antibodies, antibiotics and tissue differentiating signaling materials. Alternatively, molecular antennae may be attached to the microsphere structure to provide sites for antibody binding, acid base reaction, ionic binding, additional crosslinking, and hydrophilic/phobic receptor sites. Other non-limiting examples of additives are provided elsewhere herein.

An amount of additive is incorporated into porous microspheres 20 that will provide optimal efficacy to the subject, typically a mammal. In certain embodiments, the subject is in need of the treatment thereof. The dose and method of administration will vary from subject to subject and be dependent upon such factors as the type of mammal being treated, its sex, weight, diet, concurrent medication, overall clinical condition, the particular compounds employed, the specific use for which these compounds are employed and other factors which those skilled in the art will recognize. In certain embodiments, an additive dosage ranges from about 0.001 mg/kg to about 1000 mg/kg, such as from about 0.01 mg/kg to about 100 mg/kg or from about 0.10 mg/kg to about 20 mg/kg. The additives may be used alone or in combination with other therapeutic or diagnostic agents. Generally, treatment or management is initiated with small dosages, which can then be increased by small increments, until the desired effect under the circumstances is achieved. Additionally, one skilled in the art can rely on reference materials, such as the Physician's Desk Reference, published by Medical Economics Company at Montvale, N.J., to determine the appropriate amount of a particular drugs/agents, and hence such a dose or a lower or higher dose can be administered to a patient using the methods provided herein. In accordance with the methods provided herein, in certain embodiments, the drug is delivered to the patient (e.g., in a region of the patient) for the purposes, for example, of treating or managing a condition (i.e., a disease state, malady, disorder, etc.) in the patient. The drugs can be used as above or can be incorporated into other embodiments, such as emulsions.

### C. Pharmaceutical Compositions

Provided herein are pharmaceutical compositions comprising any of the microspheres described above and a pharmaceutically acceptable liquid or other biocompatible carrier. The compositions can be in the form of a suspension, a hydrogel, or an emulsion. The composition can also be a suspension of said microspheres in said liquid. In some embodiments, the compositions are sterile.

The pharmaceutically acceptable liquid can be, without limitation, saline, a buffer-solution, water, an isotonic solution, a biological fluid or a mixture thereof. The liquid can also be a salt solution, and, in certain embodiments, is composed of cations selected from the group consisting of sodium, potassium, calcium, magnesium, iron, zinc, and ammonium, for example, in an amount of from about 0.01 M to about 5 M.

The composition can comprise the microspheres in an amount from about 10% to about 90% by weight and the liquid (or other biocompatible carrier) in an amount from about 10% to about 90% by weight. The composition can also comprise the microspheres in an amount from about 10% to about 50% by weight and the liquid (or other biocompatible carrier) in an amount from about 50% to about 90% by weight.

Acceptable pharmaceutical carriers for therapeutic use include diluents, solubilizers, lubricants, suspending agents, encapsulating materials, solvents, thickeners, dispersants, buffers such as phosphate, citrate, acetate and other organic acid salts, anti-oxidants such as ascorbic acid, preservatives, low molecular weight (less than about 10 residues) peptides such as polyarginine, proteins such as serum albumin, gelatin or immunoglobulins, hydrophilic polymers such as poly(vinylpyrrolindinone), amino acids such as glycine, glutamic acid, aspartic acid or arginine, monosaccharides, disaccharides, and other carbohydrates including cellulose or its derivatives, glucose, mannose or dextrines, chelating agents such as EDTA, sugar alcohols such as mannitol or sorbitol, counter-ions such as sodium and/or non-ionic surfactants such as tween, pluronics or PEG.

In some embodiments, the biocompatible carrier is an aqueous-based solution, a hydro-organic solution, an organic solution, a non-aqueous solution, or a mixture thereof. In certain embodiments, the biocompatible carrier comprises a salt composed of cations, such as sodium, potassium, calcium, magnesium, iron, zinc, ammonium, and mixtures thereof, for example, in an amount of from about 0.01 M to about 5 M.

An additive (e.g., a therapeutic agent) loaded into or onto the microspheres can be released in vivo due to physiological processes. Release of the drug loaded onto the microspheres can be influenced by pH and salt concentrations. For example, drug release can be accelerated by establishing pH changes or changes in ionic strength in the environment surrounding the microspheres. Determination of such optimal drug-release conditions can easily be determined by those skilled in the art.

In some embodiments, an additive (e.g., a therapeutic agent) is released by prolonged and/or sustained release. In certain embodiments, the additive is released over a certain number of hours, days, or weeks. In one embodiment, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the drug has been released from the microsphere after a certain period of time, for example, after about 3 hours, about 6 hours, about 12 hours, about 18 hours, or after about 1 day, about 2 days, about 3 days, about 4 days, about 5 days, about 6 days, or after about 1 week, about 2 weeks, about 3 weeks, about 4 weeks, about 5 weeks, about 6 weeks, about 7 weeks, about 8 weeks, about 9 weeks, or about 10 weeks or longer. Drug release properties will depend, in part, on the properties of the specific drug used, but will be readily determinable by those skilled in the art.

In some embodiments, an additive (e.g., at therapeutic agent) is released from the microsphere over a certain number of days or weeks. In one embodiment more than about 1% but less than about 5%, about 10%, about 15%, about 20%, about 25%, about 50%, about 75% or about 90% of the drug has been released within a period of about 72 hours, a period of about 96 hours, a period of within a week, a period of within two weeks, or a period of within four weeks.

### D. Methods of Using of Bimodal Porous Microspheres

Biocompatible bimodal porous microspheres 20 according to particular embodiments provided herein may have a wide variety of medical applications. For example, the microspheres provided herein can be used for tissue engineering, tissue guided regeneration, in vivo stem cell harvesting, culturing, or differentiation, delivery and suspension of therapeutic materials in targeted human or animal tissues and/or other applications. Without being bound by any theory, the size of microspheres 20 allow them to be delivered with relative ease to virtually any target region of a living subject (e.g. by non-surgical means, such as by catheter, needle, tubing or the like) and the porous structure of microspheres 20 allow them to be of versatile application within these target regions, for example in delivering prolonged localized therapy or in promoting cell growth and transplantation.

The microspheres in the compositions and methods provided herein can be administered to (or otherwise contacted with) a tissue or organ (e.g., heart, kidney, spinal cord, uterus, liver or pancreas) by methods known in the art. In certain embodiments, the microspheres are administered (e.g., by injection) to a tissue or organ that has more than one blood supply, for example the liver, lung, spine, spinal cord, uterus or pancreas. In certain embodiments, the particle is administered to the heart, lung, nervous system, brain, lung, liver, uterus or pancreas of the patient. In some embodiments, the particle is administered to one or more blood vessels, veins or arteries comprised within the tissue or organ. In certain embodiments, the bimodal porous microspheres provided herein are used to counter ischemia in the target area, e.g., the area of administration or injection, such as in or near a tissue or organ. In some embodiments of the methods provided herein, the microspheres are administered to a patient by intraluminal administration or injection. In other embodiments of the methods provided herein, the microspheres are administered to a patient by intravascular administration or injection.

The microspheres can be delivered systemically or locally to the desired tissue or organ. In some embodiments, the microspheres can be administered to a tissue or organ before, during or after a surgery. In other embodiments, the microspheres are delivered to a tissue or organ using non-surgical methods, for example, either locally by direct injection into the selected tissues, to a remote site and allowed to passively circulate to the target site, or to a remote site and actively directed to the target site with a magnet. Such non-surgical delivery methods include, for example, infusion or intravascular (e.g., intravenous or intraarterial), intramuscular, intraperitoneal, intrathecal, intradermal or subcutaneous administration.

The diseases or disorders above can be treated or otherwise managed by administering to the patient a therapeutically effective amount of the microspheres or a pharmaceutical composition provided herein.

Administration is typically carried out by injection. In certain embodiments, the microspheres are administered by a catheter. In other embodiments, the microspheres are injected us a needle attached to a syringe. In some embodiments, administration is into a blood vessel. In other embodiments, administration is directly to the site of action, for example into a tumor mass, or into a cell, organ or tissue requiring such treatment or management. The microspheres provided herein can be administered already loaded with a drug. In other embodiments, the microspheres are administered in combination with a drug solution, wherein the drug solution is administered prior, simultaneously or after the administration of the microspheres.

When administered, the microspheres or the pharmaceutical composition are suitable for injection. In specific embodiments, the microspheres or compositions comprising the microspheres are sterile. The microspheres may be sterilized by any method known in the art, for example, by irradiation, such as gamma or beta irradiation. In certain embodiments, the microspheres are prepared aseptically using aseptic techniques. In some embodiments, the microspheres prepared aseptically comprise an additive, such as a therapeutic agent or drug.

In certain embodiments, provided herein are compositions and methods suitable for treating or otherwise managing tumors or other cancers, non-tumorigenic angiogenesis-dependent diseases, or pain, such as pain related to the presence of a tumor or other cancer, or a symptom thereof. Such cancers include, without limitation (both anatomically and by primary neoplastic site), liver, ovarian, breast, kidney, lung, pancreatic, thyroid, prostate, uterine, skin cancer, head and neck tumors, breast tumors, brain, bone, soft tissues (such as sarcoma, lipoma, malignany fibrous histiocytoma), blood (such as lymphoma), Kaposi's sarcoma, and superficial forms of bladder cancer. In certain embodiments, the method of treatment or management may be the result of localized (or systemic) drug delivery released from the drug-loaded microspheres, either alone or in combination with embolic effects of the microspheres. In certain embodiments, drug-loaded microspheres provided herein are administered to a site-specific location other than a blood vessel (e.g., directly into a tumor mass), and no vessel embolization occurs.

In addition to cancer, however, numerous other non-tumorigenic angiogenesis-dependent diseases which are characterized by the abnormal growth of blood vessels can also be treated, either via down-regulation or up-regulation, or otherwise managed with the microspheres or pharmaceutical compositions provided herein. Representative examples of such nontumorigenic angiogenesis-dependent diseases include, without limitation, hypertrophic scars and keloids, proliferative diabetic retinopathy, rheumatoid arthritis, arteriovenous malformations, lymphangitic malformations, venous malformations, atherosclerotic plaques, delayed wound healing, hemophilic joints, nonunion fractures Klippel Trenaunay Syndrome, Parkes Weber Syndrome, Osler-Weber-Rendu Syndrone, Blue Rubber Bleb Syndrome, cutnaoues and subcutaneous nevi, hemangiomas, leiomyomata, adenomas, hamartomas, psoriasis, pyogenic granuloma, scleroderma, tracoma, menorrhagia and vascular adhesions.

Similarly, the microspheres and compositions provided herein can be used to deliver drugs to various cells, tissues or organs in need thereof. For example, the microspheres and compositions can be used to treat or otherwise manage tumors or cancers, inflammatory diseases or other diseases associated with inflammation, or symptoms thereof. In other embodiments, the microspheres and compositions provided herein can be used to treat or otherwise manage uterine fibroids.

In some embodiments, a drug or therapeutic agent can be administered to a tissue, organ or cell prior to administration of microspheres. In certain embodiments, a drug or therapeutic agent is administered between about 1 minute and about 60 minutes prior to administration of microspheres. In some embodiments, a drug or therapeutic agent is administered to a tissue, organ or cell within 1, 5, 10, 15, 20, 30, 45 minutes or about 1, 2, 4, 6, 10, 12, 18, 20 or 24 hours of administration of microspheres. In yet other embodiments, a drug or therapeutic agent is administered concurrently with microspheres. In certain embodiments, microspheres are administered to a tissue, organ or cell prior to administration of the a drug or therapeutic agent. In certain embodiments, microspheres are administered between about 1 minute and about 60 minutes prior to administration of a drug or therapeutic agent. In some embodiments, microspheres are administered to a tissue, organ or cell within 1, 5, 10, 15, 20, 30, 45 minutes or about 1, 2, 4, 6, 10, 12, 18, 20 or 24 hours of administration of a drug or therapeutic agent.

In addition, microspheres (with out without bioactive additives) may be administered simultaneously with cell delivery, which may include without limitation pancreatic islet cell transplantation for diabetes, stem cell administration for myocardial synthesis or preservation, bone promotion or synthesis within osseous structures, and catheter based stem cell administration to liver or lung.

In some embodiments, cells (e.g., stem cells) can be administered to a tissue, organ prior to administration of microspheres. In certain embodiments, cells (e.g., stem cells) are administered between about 1 minute and about 60 minutes prior to administration of microspheres. In some embodiments, cells (e.g., stem cells) are administered to a tissue or organ within 1, 5, 10, 15, 20, 30, 45 minutes or about 1, 2, 4, 6, 10, 12, 18, 20 or 24 hours of administration of microspheres. In yet other embodiments, cells (e.g., stem cells) are administered concurrently with microspheres. In certain embodiments, microspheres are administered to a tissue or organ prior to administration of the cells (e.g., stem cells). In certain embodiments, microspheres are administered between about 1 minute and about 60 minutes prior to administration of cells. In some embodiments, microspheres are administered to a tissue or organ within 1, 5, 10, 15, 20, 30, 45 minutes or about 1, 2, 4, 6, 10, 12, 18, 20 or 24 hours of administration of cells (e.g., stem cells). In these various embodiments, cells and/or microspheres can be administered to a tissue or organ optionally with a drug or therapeutic agent.

An effective dose of cells for use in the methods provided herein will vary depending on the cell type used and/or the delivery site, and such doses can be readily determined by a physician. In certain embodiments, the number of cells, is in the range of 1×10⁵ to 1×10⁹. For example, cells can be administered in a dose between about 1×10⁶ and 1×10⁸, such as between 1×10⁷ and 5×10⁷. Depending on the size of the organ or tissue to be delivered, for example, or the size of the damaged area, more or less cells can be used. For example, a larger region of damage may require a larger dose of cells, and a small region of damage may require a smaller does of cells. On the basis of body weight of the recipient, an effective dose may be between 1×10⁵ and 1×10⁹ per kg of body weight, such as 1×10⁵, 1×10⁶, 1×10⁷, 1×10⁸, or 1×10⁹ (or any range thereof) per kg of body weight, for example between 1×10⁶ and 5×10⁶ cells per kg of body weight. Patient age, general condition, and immunological status may be used as factors in determining the dose administered, and will be readily determined by the physician.

Further non-limiting, exemplary areas of application of the compositions and methods provided herein, which illustrate the potential functionality of microspheres 20, are intraarterial brachytherapy, islet cell transplantation and others described below.

### 1. Intraarterial Brachytherapy

Intraarterial brachytherapy is a form of radiotherapy involving catheter-based infusion of radioactive materials through an artery to a target area within the body, typically for treatment or management of cancerous tissues or a symptom thereof. The delivered radioactive materials may have an embolic effect (blocking off blood supply to the target area), which may be beneficial in maintaining the therapy in the target area. However, such localized radiotherapy is typically dependent on generation of free radicals, and in particular oxygen free radicals. Generation of oxygen free radicals may be promoted by providing an oxygenated environment around the target area. Thus, complete embolization in the target area may not be desirable since blood flow is required to provide oxygen. In fact, a well oxygenated environment can result in increased radical generation, translating into a correspondingly increased radiotherapeutic effect. Given these requirements, porous microspheres 20 according to embodiments provided herein may provide a well suited material for intraarterial brachytherapy because they are capable of producing a local regional source of radioactivity (remaining in the target area) while allowing continued blood inflow (perfusion) to the target area and a minimal or optimal embolic effect. Furthermore, addition therapeutic agents may be incorporated into the microsphere structure for prolonged delivery to the target area. Microspheres 20 may be made radioactive by various means, such as by coating them with a resin and/or subsequently bombarding microspheres 20 with radiation. Microspheres 20 may also be fabricated to incorporate radiopharmaceutical by covalent bonding to any of the materials used to form microspheres 20 (as described in U.S. patent application Ser. No. 10/762,507) or by the techniques described in U.S. Pat. No. 5,011,677.

### 2. Islet Cell Transplantation

Another exemplary application for microspheres 20 is in the field of islet cell transplantation. Islet cell transplantation typically provides insulin-producing islet cells from a donor pancreas to a diabetic subject unable to produce insulin. Typically, a load of islet cells are implanted into the portal vein of the recipient's liver through catheter-based infusion. Current drawbacks to this technique are that the transplanted cells are injected as a suspension, which may result in cell to cell contact, increased compression of the cells, increased perfusion pressures, and local inflammatory reaction; all factors which contribute to apoptosis and transplant rejection. The likelihood of transplanted cells surviving may be increased if cells are sufficiently spaced apart to avoid or reduce cell to cell contact, sufficient blood flow is maintained, and bioactive substances are administered on a local level. Porous microspheres 20 according to embodiments of provided herein, co-administered at the time of transplantation (with or without bioactive substances, and with or without the ability to be completely bioabsorbable), may result in decreased cell density and may allow for continued perfusion, thereby increasing the probability of transplant survival. Specifically, the macropore structure within the microsphere may be designed to provide a scaffolding suitable for holding the islet cells a suitable distance apart, while the bimodal pore structure would permit suitable blood flow dynamics in and around the cells.

### 3. Other Cell Delivery and Tissue Regeneration

The qualities of microspheres 20 that make them suitable for islet cell transplantation (e.g., cell separation and continued perfusion) also make them potentially suitable for many other types of cell growth, cell transplantation and tissue regeneration applications. For example, microspheres 20 according to embodiments provided herein may be used in stem cell therapy acting as an injectable scaffolding for supporting stem cell differentiation and tissue genesis. Injection of microspheres 20 (with bioactive agents such as selectins, hormones, cell receptors, viruses, and pharmaceuticals that may be doped, bound or mixed with the sphere surface or substrate) may result in stem cell or targeted cell migration (for harvesting, processing, or differentiation into terminal cell lines) or in specific embodiments, in vitro creation of functional cell groups, or organs (organogenesis). Particular non-limiting stem cell therapy applications include injection of microspheres 20 and stem cells into the liver or lungs which have unique anatomic characteristics—i.e. the liver has vascular inflow through both arterial supply and portal inflow and the lung has a dual inflow blood supply through the pulmonary artery and the bronchial arteries for concentration, harvesting or differentiation. Furthermore, such microspheres 20 may be coated or doped with various promoters, chemoattractants or cell potentiators that may promote cellular migration and/or differentiation and may facilitate establishment of local tissue regeneration in target areas.

Microspheres 20 comprising a base polymer that is bioabsorbable (or bioerodable or biodegradable) may have further advantages. In the context of cell transplantation or tissue generation, such microspheres 20 may decompose over time, leaving behind only the generated or transplanted cell structure. Decomposable microspheres 20 may also allow for increased blood flow to delivered therapies and increased penetration of therapies into target tissues. The gradual breakdown of microspheres 20 may also allow for the gradual delivery of localized therapy (such as drug therapy or radiation therapy) to the target area.

In certain embodiments, provided herein are methods of tissue construction and generation. In some embodiments, the method comprises administering a composition of bimodal porous microspheres, optionally in a biocompatible carrier, to a patient, such as a mammal.

The tissue construction and generation methods provided herein provides the advantage of not being limited to the repair of any specific type of tissues or tissue defect in any specific organ or body part. Rather, the method is suitable for the construction and generation of defective tissues on any kind and of any parts of the body, including, but not limited to, heart, coronary vessels, blood vessels, spinal cord, bone, cartilage, tendon, ligament, breast, liver, gallbladder, bile duct, pancreas, intestinal tissues, urinary system, skin, hernia, and dental tissues. Further, in certain embodiments, the use of a composition comprising a bimodal porous microsphere and a cell, such as a stem cell (e.g., a pluripotent mesenchymal stem cell) can improve tissue acceptance and the effectiveness of the treatment. The methods provided herein can also increase connective tissue response.

Provided herein is a method of tissue (or organ) construction or regeneration in a patient, comprising administering cells, such as stem cells, to a patient. In some embodiments, the cells are contacted with the tissue or organ. In certain embodiments, the patient is administered an injectable composition. The injection can be carried out by conventional syringes and needles of 9 to 26 gauge. The injection can also be facilitated by various techniques such as endoscopic delivery or laparoscopic technique. Furthermore, when combined with the various advantageous embodiments of the injectable composition, such as autologous cells and therapeutic agents, the methods provided herein can offer additional and more beneficial therapeutic effects to further improve the tissue construction and generation.

The frequency and the amount of injection using the methods provided herein is determined based on the nature and location of the particular case of the tissue or organ defect being treated. In certain embodiments, multiple injections are not necessary. In other embodiments, however, repeated injection may be necessary to achieve optimal results. A skilled practitioner can determine the frequency and the amount of the injection for each particular case.

In certain embodiments, after administration, the microspheres become secured at the position of the injection and are not digested or eliminated by the lymphatic system, and/or the microspheres are not displaced from the position of injection. In other embodiments, the microspheres are bioabsorbable or biodegradable.

Properties of certain bimodal porous microspheres provided herein allow the microspheres to provide a scaffold for effective tissue construction, tissue generation, and tissue engineering. The ability of forming a scaffold at the injection site makes the microspheres provided herein particularly effective in providing tissue repair. The size of the scaffold can be determined by the amount and frequency of the injection, which is in turn determined by the nature and location of the tissue construction and generation being performed. A skilled practitioner would be able to determine the exact amount and frequency of injection for each particular case.

The combination of the scaffold effect with the fact that microspheres provided herein can comprise cells, such as stem cells (e.g., mesenchymal stem cells) can promote new cell growth at the site of injection, makes the methods provided herein particularly effective in providing a mechanism for tissue construction and generation. Since, in certain embodiments, microspheres provided herein are bioabsorbable and/or biodegradable, they can be incorporated into the repaired tissue after serving as scaffold for the tissue generation.

In certain embodiments of the methods provided herein, tissue construction and generation is accomplished by administering the microspheres comprising cells extra corporeally into organs, components of organs, or tissues prior to their inclusion into the body, organs, or components of organs.

The methods provided herein can be carried out by any type of sterile needles, e.g., from 9 to 26 gauge, and corresponding syringes or other means for injection, such as a three-way syringe. The needles, syringes and other means for injection are commercially available from suppliers such as VWR Scientific Products (West Chester, Pa.), Beckton Dickinson, Kendal, and Baxter Healthcare. The size of the syringe and the length of the needle used will dependent on the particular injection based on factors such as the specific disease or disorders being treated, the location and depth of the injection, and the volume and specific composition of the injectable suspension being used. A skilled practitioner will be able to make the selection of syringe and needle based on experience and the teachings provided herein.

In one embodiment, a method of preparing an injectable suspension for use in the compositions and methods provided herein is as follows. Bimodal porous microspheres are washed, sterilized, and then mixed with cell culture containing cells, such as stem cells (e.g., mesenchymal stem cells). The cells are then detached from their original culturing surface, such as by trypsinization. The mixture of microspheres, cell culture medium and detached cells is allowed to continue a culturing process that is both sterile and suitable for stem cell culturing for a period of no less than 12 hours. The suspension is then ready for injection.

### 4. Gene Therapy

In some embodiments of the compositions and methods provided herein, the bimodal porous microspheres comprise cells, such as stem cells. In certain embodiments, the cells are not human embryonic stem cells. In some embodiments, the cells are genetically engineered to express one or more polypeptides, such as a therapeutic agent, using techniques known in the art. In some embodiments, the cell comprises a nucleotide sequence that expresses the polypeptide, e.g., in a vector. In certain embodiments, the cell expresses the polypeptide continuously. In other embodiments, the cell express the polypeptide transiently. In yet other embodiments, the cell can be regulated to express the polypeptide, e.g., by the use of an inducible promoter or other regulatory element in the vector comprising the nucleic acid sequence encoding the polypeptide. In other embodiments, the bimodal porous microspheres comprise genetic material.

Genetic material comprising nucleic acids, polynucleotides, RNA and DNA, of either natural or synthetic origin, including recombinant RNA and DNA and antisense RNA and DNA; hammerhead RNA, ribozymes, antigen nucleic acids, both single and double stranded RNA and DNA and analogs thereof, either in combination or not with other elements such as, for example, without limitation, tissue specific enhancers, and nuclear localization signals, can be introduced into eukaryotic cells via conventional transformation or transfection techniques. As used herein, the terms “transformation” and “transfection” are intended to refer to a variety of art-recognized techniques for introducing foreign nucleic acid into a host cell, including, for example, without limitation, calcium phosphate or calcium chloride co-precipitation, DEAE-dextran-mediated transfection, lipofection, or electroporation. Suitable methods for transforming or transfecting host cells can be found in Sambrook et al. (2001) Molecular Cloning: A Laboratory Manual, Cold Spring Harbor Laboratory Press, Cold Spring Harbor, N.Y., and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Selectable markers can include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

In order to obtain an efficient in vivo transfer of the therapeutic agents, various transfection agents are employed. Representative examples of transfection agents which are suitable for use with the methods provided herein include, without limitation, calcium phosphate or calcium chloride co-precipitation, DEAE-dextran-mediated transfection, quaternary ammonium amphiphile DOTMA ((dioleoyloxypropyl)trimethylammonium bromide, commercialized as Lipofectin by GEBCO-BRL))(Felgner et al, (1987) Proc. Natl. Acad. Sci. USA 84, 7413-7417; Malone et al. (1989) Proc. Natl. Acad. Sci. USA 86 6077-6081); lipophilic glutamate diesters with pendent trimethylammonium heads (Ito et al. (1990) Biochem. Biophys. Acta 1023, 124-132); the metabolizable parent lipids such as the cationic lipid dioctadecylamido glycylspermine (DOGS, Transfectam, Promega) and dipalmitoylphosphatidyl ethanolamylspermine (DPPES)(J. P. Behr (1986) Tetrahedron Lett. 27, 5861-5864; J. P. Behr et al. (1989) Proc. Natl. Acad. Sci. USA 86, 6982-6986); metabolizable quaternary ammonium salts (DOTB, N-(1-[2,3-dioleoyloxy]propyl)-N,N,N-trimethylammonium methylsulfate (DOTAP)(Boehringer Mannheim), polyethyleneimine (PEI), dioleoyl esters, ChoTB, ChoSC, DOSC)(Leventis et al. (1990) Biochim. Inter. 22, 235-241); 3beta[N—(N′,N′-dimethylaminoethane)-carbamoyl]cholesterol (DC-Chol), dioleoylphosphatidyl ethanolamine (DOPE)/3beta[N—(N′,N′-dimethylaminoethane)-carbamoyl]cholesterolDC-Chol in one to one mixtures (Gao et al., (1991) Biochim. Biophys. Acta 1065, 8-14), spermine, spermidine, lipopolyamines (Behr et al., Bioconjugate Chem, 1994, 5: 382-389), lipophilic polylysines (LPLL) (Zhou et al., (1991) Biochim. Biophys. Acta 939, 8-18), [[(1,1,3,3-tetramethylbutyl)cresoxy]ethoxy]ethyl]dimethylbenzylamionium hydroxide (DEBDA hydroxide) with excess phosphatidylcholine/cholesterol (Ballas et al., (1988) Biochim. Biophys. Acta 939, 8-18), cetyltrimethylammonium bromide (CTAB)/DOPE mixtures (Pinnaduwage et al., (1989) Biochim. Biophys. Acta 985, 33-37), lipophilic diester of glutamic acid (TMAG) with DOPE, CTAB, DEBDA, didodecylammonium bromide (DDAB), and stearylamine in admixture with phosphatidylethanolamine (Rose et al., (1991) Biotechnique 10, 520-525), DDAB/DOPE (TransfectACE, GIBCO BRL), and oligogalactose bearing lipids (Remy et al., to be published).

Various transfection enhancer agents can also be to increase the efficiency of transfer of the bioactive therapeutic factor into cells. Suitable transfection enhancer agents include, for example, without limitation, DEAE-dextran, polybrene, lysosome-disruptive peptide (Ohmori N I et al., Biochem Biophys Res Commun Jun. 27, 1997; 235(3):726-9), chondroitan-based proteoglycans, sulfated proteoglycans, polyethylenimine, polylysine (Pollard H et al. J Biol Chem, 1998 273 (13):7507-11), integrin-binding peptide CYGGRGDTP, linear dextran nonasaccharide, glycerol, cholesteryl groups tethered at the 3′-terminal internucleoside link of an oligonucleotide (Letsinger, R. L. 1989 Proc Natl Acad Sci USA 86: (17):6553-6), lysophosphatide, lysophosphatidylcholine, lysophosphatidylethanolamine, and 1-oleoyl lysophosphatidylcholine. In certain embodiments, suitable transfection agents include, without limitation, lipopolyamines as disclosed in U.S. Pat. No. 5,171,678, issued to Behr, et al., Dec. 15, 1992, U.S. Pat. No. 5,476,962 issued to Behr, et al., Dec. 19, 1995, and U.S. Pat. No. 5,616,745 issued to Behr, et al., Apr. 1, 1997, the entire disclosures of which are incorporated herein by reference in their entirety.

In other embodiments, the microspheres provided herein comprise an expression vector. In other embodiments, the microspheres comprise a cell that comprises an expression vector. The expression vector can contain a nucleic acid encoding a therapeutic agent or polypeptide (or a portion thereof). As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Specific examples of viral vectors, include, without limitation, adenovirus and retrovirus vectors for gene therapy using the microspheres and transfection agents provided herein. Also contemplated is the use of a virus-like particle containing a bioactive therapeutic factor, wherein the virus-like particle is physically linked to the transfection agent, which is also linked to the microparticle. Such virus-like particles may be designed using polyethylenimine (PEI) conjugated to the integrin-binding peptide CYGGRGDTP via disuphide bridge formation. Such PEI/RGD-containing peptide/complexes share with adenovirus constitutive properties such as size and a centrally protected core, as well as early properties, such as cell entry mediated by integrins and acid-triggered endosome escape (Erbacher et al., to be published).

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, other forms of expression vectors are also contemplated, such as viral vectors (e.g., replication defective retroviruses, adenoviruses and adeno-associated viruses), which serve equivalent functions.

The recombinant expression vectors used herein can comprise a nucleic acid in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to-mean that the nucleotide sequence of interest is linked to-the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g. polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors can be introduced into host cells to thereby produce proteins or peptides, including fusion proteins or peptides, encoded by nucleic acids as described herein.

The recombinant expression vectors can be designed for expression of a polypeptide in prokaryotic (e.g., E. coli) or eukaryotic cells (e.g., insect cells (using baculovirus expression vectors), yeast cells, or mammalian cells). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

In another embodiment, a nucleic acid is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see Sambrook et al. (2001) Molecular Cloning: A Laboratory Manual, Cold Spring Harbor Laboratory Press, Cold Spring Harbor, N.Y.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43 235-275), in particular promoters of T cell receptors (Winoto and Baltimore (1989). EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), prostate-specific promoters and/or enhancers (U.S. Pat. Nos. 5,830,686, and 5,871,726, the entire of which are incorporated herein by reference in their entirety) and mammary gland-specific promoters (e.g., milk whey promoter, U.S. Pat. No. 4,873,316 and European Application Publication No. 264,166). Developmentally-regulated promoters are also encompassed, for example the murine hox promoters (Kessel and Gruss (1990) Science 249:374-379) and the α.-fetoprotein promoter (Campes and Tilghman (1989) Genes Dev. 3:537-546).

In certain embodiments, the recombinant expression vector comprises a DNA molecule cloned into the expression vector in an antisense orientation. That is, the DNA molecule is operably linked to a regulatory sequence in a manner which allows for expression (by transcription of the DNA molecule) of an RNA molecule which is antisense to the mRNA encoding a given polypeptide.

Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

In one embodiment, a cancer may be treated by supplying a toxin gene on a DNA template with a tissue specific enhancer and/or promoter to focus expression of the gene in the cancer cells. For example, toxin genes include, without limitation, the diphtheria toxin gene. Intracellular expression of diphtheria toxin is known to kill cells. The use of certain promoters could be tissue-specific such as using a pancreas-specific promoter for pancreatic cancer. Thus, a functional diphtheria toxin gene delivered to pancreatic cells could, in theory, eradicate the entire pancreas. This strategy could be used as a treatment for pancreatic cancer. The tissue specific enhancer would ensure that expression of diphtheria toxin would only occur in pancreatic cells. DNA/lipopolyamine/microsphere complexes containing the diphtheria toxin gene under the control of a tissue specific enhancer would be introduced directly into a cannulated artery feeding the pancreas. The infusion would occur on some dosing schedule for as long as necessary to eradicate the pancreatic tissue. Other lethal genes besides diphtheria toxin could be used with similar effect, such as genes for ricin or cobra venom factor or enterotoxin.

Another specific example would be the use of prostate specific antigen promoter/enhancer to direct an additive, such as a therapeutic agent, to the prostate of a patient in need of treatment for prostatic cancer. One could also treat specialized cancers by the transfer of genes such as, for example, without limitation, the p53 gene, the retinoblastoma gene (and others of that gene family) that suppress the cancer properties of certain cancers.

In certain embodiments of the compositions and emthdos provided herein related to islet cell transplantation; islet cells are modified by gene therapy. For example, methods of modifying islet cells through gene therapy approaches have been described to protect cells from apoptosis (see, e.g., Tellez et al. (2005) Gene Ther. 12:120-128; Giannoukakis et al. (1999) Diabetes 48:1730-1736), induce islet cell proliferation or augment directly the function of the transplanted tissue to promote disease treatment with fewer donor cells (see, e.g., Rao et al. (2004) Expert Opin. Biol. Ther. 4:507-5518; Lopez-Talayera et al. (2004) Endocrinol. 145:467-474)

Various non-limiting examples of potential applications of porous microspheres 20 are described further below.

### 5. Sequestration of Cells

Microspheres 20 may be administered in vivo with active cell lines, or may act as a sieve to extract cells from the bloodstream for sequestration or differentiation due to the natural filtration of blood as it flows (perfuses) through microsphere 20. Such perfusion through microsphere 20 may serve to decrease blood clot formation. Doping, coating or simultaneous administration of promoters, chemo attractants or cell potentiators may promote cellular migration and/or differentiation that could establish the bases for local tissue regeneration in solid organs, bone and cartilage, mucosa, endothelium, nervous, endocrine, or hematogenous tissues/cell lines for the purposes of tissue regeneration, differentiation, or altering the cellular constituent within a specific anatomical or histological environment.

### 6. Chemoembolization/Radioembolization

Chemoembolization is a combination of chemotherapy and embolization or embolotherapy (as described above), used typically to treat cancer. Similarly, radioembolization is a combination of radiation therapy and embolization or embolotherapy. Microspheres 20 may be injected to a target area as a standalone therapy or for the purposes of interspersion between terminal therapeutic embolics to allow for gradual migration of the embolic into tumor blood supply, while providing continued perfusion/blood flow into targeted tumor. The addition of chemotherapeutics to the microsphere matrix may increase the efficacy of the therapy by improving the timing of exposure of therapy with the terminal embolic effect of embolic material.

### 7. Biogenerator Medium

Biogenerators are devices used for growing cells in vitro (external to a living body). Without being bound by any theory, the increased spacing that could be provided within a biogenerator through implementation of biocompatible (bioabsorbable or permanent) microspheres 20 could increase surface area for agitation, serve as binding sites and crypts for cellular ingrowth, and also decrease cell to cell contact which are all desirable criteria in biogenerator media.

### 8. Delivery of Imaging Agents/Localizers

Microspheres 20 comprising or containing iodine-impregnated polymers such as those described in PCT application PCT/US98/23777 may hold application for determination of true vascularity ratios of tumor or organ perfusion, as an alternative to Tc-99MAA, which has proven to be suboptimal due to its emulsified nature in applications of liver directed therapy. Other non-limiting examples of tracers or imaging agents that may be added to microspheres 20 include radiolabelled antibodies, FDG, iondinated contrasts, ferromagnetic agents (such as small particle iron oxide (SPIO)), gadolinium chelates, magnesium, barium, or various diagnostic and/or therapeutic radioonucleoides.

9. Cosmetic therapy/Topical and transdermal medication delivery

In certain embodiments, porous microspheres 20 may provide a vehicle for prolonged, controlled delivery of a large variety of topical and transdermal cosmetic substances such as fragrances, emollients, sunscreens, and anti-inflammatory, antifungal and antimicrobial agents, as described by Smith et al. in “The characteristics and utility of solid phase porous microspheres: a review”, Journal of Drugs in Dermatology, November-December, 2006. Incorporating such additives into microspheres 20 may decrease direct contact of the cosmetic additive with the surrounding tissue and thereby increase the metabolic half-life of the additive. Localized intraluminal (vascular and nonvascular), interstitial, subdermal, transdermal, or subcutaneous injection and fixation of microspheres 20 may create a scaffolding for the administration of viscous materials for increased localized bioavailability and prolonged exposure. Interporous distance may affect the degree of resorption and the local inflammatory reaction. Simultaneous injection with hyaluronidase and the like may decrease resorption rate, and thus prolong efficacy of therapy.

### 10. Cell Viability and Storaze

Without being bound by any theory, admixing cells with inert biocompatible porous microspheres 20 may allow for more efficient storage and maintenance of ex vivo cell lines than single slide methods which are currently in use. Microspheres 20 may provide a suitable scaffolding or matrix within which the stored cells may be efficiently and safely packed.

### 11. Tissue Harvesting/Tissue Expansion/Organ and Tissue Engineering

In certain embodiments, a mixture of microspheres 20 and stem cells or non differentiated organ cell lines, in addition to promoters, may be molded or formed into shapes that may serve as a basic functional unit for organ and tissue engineering.

While a number of exemplary aspects and embodiments have been discussed above, those of skill in the art will recognize certain modifications, permutations, additions and sub-combinations thereof. By way of non-limiting example, embodiments of microspheres 20 may also be applied in:
improving uptake of bone stem cells into heart tissue;
producing a birth control pill based upon mediated sustained hormonal response;
treatment of hypothyroidism, or a symptom thereof, through transdermal injection;
regeneration of lung tissue in compromised patients (including creation of new functional lung tissue, blood vessels, or means for improved oxygen exchange);
regeneration of nervous tissue and brain tissue for neurological disease or trauma (such as Alzheimer's, Parkinson's, brain damage, spinal cord injury);
activation of local angioneogenisis for accelerated would healing (i.e. growing blood vessels in injured area).
producing a powder to be applied in trauma situations to provide sustained antibiotics and emergency thombogenesis (i.e. forming of blood clots to prevent extreme bleeding).

### E. Kits

Also provided herein are pharmaceutical packs and kits comprising one or more containers filled with one or more of the ingredients of the aforementioned microspheres and compositions provided herein. The kits can comprise or more of microspheres, a contrast agent, and solution comprising one or more drugs, wherein one, two, three or more of the components can be in one, two, three or more vials. Associated with such container(s) can be instructions for use and/or a notice in the form prescribed by a governmental agency regulating the manufacture, use or sale of pharmaceuticals or biological products, reflecting approval by the agency of the manufacture, use or sale of the product for patient (e.g., human or other mammal) administration. The reagents of any of the assays or methods described herein can also be included as components of a kit.

In one kit format, the microspheres provided herein are present in a liquid, physiologically compatible solution in one vial. In another kit format, the microspheres provided herein can be provided in dry form in one vial and the drug solution and contrast agent can be provided in a second and/or optionally a third vial. In certain embodiments, the microsphere comprising the contrast agent are present in one vial, and the drug is present in solution in another vial. In this form, the contents of the two vials can be mixed together prior to or concurrently with administration. In other embodiments, the microspheres comprising the contrast agent and the drug are provided in dry form in one vial. The powder can then be suspended in a suitable liquid prior to administration or a second vial is provided, which contains the injectable solution and the contents of both vials are combined prior to administration or concurrently with administration.

Finally, in another kit format the microspheres provided herein are present in one vial and a second vial contains a pharmaceutically acceptable solution comprising the contrast agent. The microspheres in the first vial can be pre-loaded with a drug, or the drug solution can optionally be present in a third vial. The microspheres can then be mixed together with the drug solution and/or contrast agent, for example, prior to or concurrently with administration.

The following examples are offered by way of illustration, and not by way of limitation.

### EXAMPLES

The practice of the invention employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Current Protocols in Immunology, John Wiley & Sons (1987 and annual updates); Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

### Example 1

### Preparation of Bimodal Porous Microspheres

The microspheres are prepared by the following method using a bimodal porous microsphere fabricating apparatus. Briefly, 0.2 g of Poly(DTE carbonate) is stirred and dissolved in the mixture of 3 mL 1,4-dioxane and 0.3 mL water to form a homogenous solution in the storage vessel. The solution is subsequently poured into the chamber of the injector. 7 gram of sodium chloride is added to the solution via a conduit connected to the injector. A pressure is then applied to the injector chamber to allow injection of solution droplets into the quenching tower. The droplets are quickly frozen and solidified as soon as they enter into the tower, leaving bimodal porous structures. Next, residual salt and solvents are washed off from microspheres. The washing process is repeated several times until the silver nitrate test shows no additional release of chloride ions into the water. The resulting microspheres are removed from the water and dried to constant weight.

### Example 2

### Assessment of Microsphere Morphology By SEM Scanning Electron Microscopy

SEM scanning electron microscopy is performed to assess the morphology of microspheres. Briefly, samples are prepared by cryofracture of the microspheres in liquid nitrogen. The microspheres are submitted to a series of pressurization-depressurization to ensure the filling of the pores with water. Next, samples are dried under vacuum, mounted on metal stubs using adhesive tabs. They are coated with silver using a sputter coater. An Hitachi S450 SEM at 15 kV is used for examination.

The sizes of the pores of the digital images obtained with the SEM are analyzed with the NIH Image 1.6 software. Evaluated image parameters include pore area, perimeter, major and minor axis of the ellipse. Adjustment of the digital images is made prior to pore assessment. The numbered pores are compared with the actual digital image to confirm pore location. Certain pore numbers which are not properly represented are excluded from the statistical data analysis. For each microsphere, at least 3 different digital images at 2 different magnifications (low magnification (scale bar of 200 μm) and high magnification (scale bar of 10 μm)), are analyzed.

### Example 3

### Assessment of Pore Volume and Size Distribution

Microspheres are analyzed as the macropore spacer material is still inside the polymer matrix. The pore volume and the pore size distribution are determined by recording mercury intrusion volume into the microspheres at different pressures with a mercury porosimeter. The filling pressure is recorded up to 3,000 psia. This pressure corresponds to the energy required to intrude mercury into pores of 0.06 μm or larger. The pore diameter and porosity values refer to equivalent cylindrical pores with a diameter smaller than 310 μm.

These values are determined from the Washburn equation:

D=−(1/P)4γ cos f

wherein D is the pore diameter in microns; P is the applied pressure (psia); γ is the surface tension between mercury and the scaffold surface (dynes/cm); and φ is the contact angle.

The values recommended for the surface tension and the contact angles are:

g=485 dynes/cm

φ=130°

### Example 4

### Exemplary Preparation of Bimodal Porous Trisacryl Microspheres

In a beaker containing 100 ml of demineralized water, 58 g of sodium chloride and 27 g of sodium acetate are dissolved. 400 ml of glycerol is added and then the pH is adjusted between 5.9 and 6.1. Then 90 g of N-tris-hydroxy-methyl methylacrylamide, 35 mg of diethylaminoethylacryl-amide, 10 g of N,N-methylene-bis-acrylamide, as well as 1-50 g of insoluble microparticles (or other suitable macropore spacer material) having a diameter between 20 and 500 μm and molecular weight of 50-70 g/mol are added. One heats at 60-70° C. and 100 mo of a hot 300 mg/ml gelatin solution is added. The total volume of the mixture is adjusted to 980 ml by addition of hot water and then 20 ml of a 70 mg/ml ammonium persulfate solution and 4 ml of N,N,N′,N′-tetramethylethylenediamine are added.

This solution is poured into paraffin oil at 50-70° C. stirring. After a few minutes, the polymerization reaction of acrylic monomers is manifested by an increase of temperature. The macropore spacer material is then washed off or otherwise removed from the microspheres using the methods provided herein. The microspheres are then recovered by decanting, washed carefully, screened and sterilized in an autoclave in a buffered medium.

Those microspheres, after screen calibration, possess the characteristics useful for embolization, including a marked cationic charge and an effective adhesion agent (gelatin or denatured collagen).

### Example 5

### Exemplary Preparation of Bimodal Porous Microspheres Comprising 5-20% Polyvinyl Alcohol

Five to twenty grams of polyvinylalcohol are dissolved in 75 ml of a 0.5 M H₂SO₄-0.1 M NaCl solution under stirring. The suspension is agitated until a clear solution forms and 1-50 g of insoluble microparticles having a diameter between 20 and 500 μm and molecular weight of 50-70 g/mol (or other suitable macropore space material) and 25 ml of formalaldehyde are then added to the solution. The resulting mixture is rapidly poured into 500 ml of agitated paraffin oil containing 2% of sorbitan sesquioleate. Under these conditions, an emulsion is formed with droplets of polyvinylalcohol in suspension oil. The emulsion is heated at about 80° C. for at least 6 hours to obtain the condensation of formaldehyde on polyvinylalcohol chains and thus forming spherical particles of crosslinked polyvinylalcohol.

Particle size is managed by the speed of agitation of the emulsion. For example, in order to obtain microspheres with diameter around 300 μm (average dimension), the agitation speed is kept at about 250 rpm.

Hydrogel microspheres of polyvinylalcohol are then collected by filtration. Alternatively, hydrogel microspheres of polyvinylalcohol may be collected by centrifugation or by simple decanting. The macropore spacer material is then washed off or otherwise removed from the microspheres using the methods provided herein. Residue oil is extracted by non-polar solvents or chlorinated solvents such as methylene chloride. The resulting oil-free microspheres are then treated with a 0.5 M Tris-HCl buffer (pH 9) overnight at room temperature to neutralize excess aldehydes.

Finally, the polyvinylalcohol microspheres are washed with physiological aqueous buffers, sieved to desired diameter, sterilized and stored as liquid suspensions. This material can be used for embolization procedure.

### Example 6

### Exemplary Preparation of Bimodal Porous Microspheres Comprising a Sodium Acrylate and Vinyl Alcohol Copolymer

One-half of a gram of benzoyl peroxide as a polymerization initiator and 1-50 g of NaCl (or 1-50 g of insoluble microparticles having a diameter between 20 and 500 μm and molecular weight of 50-70 g/mol; or other suitable macropore space material) are added to 60 g of vinyl acetate and 40 g of methyl acrylate. This is dispersed in 300 ml of water containing 3 g of partially saponified polyvinylalcohol as a dispersion stabilizer and 10 g of NaCl. The suspension polymerization is carried at 65° C. for 6 hours. After removing the solvent, the polymer is dried for 24 hours in a freeze dryer. Twenty grams of the dried powder is suspended in a saponification fluid containing 200 g of methanol and 10 g of water. Then 40 ml of 10 N NaOH solution was added drop wise by maintaining the reaction at 10° C., and then the reaction was carried out at 30° C. for 24 hours. After the saponification reaction is completed, the reaction product is washed with methanol, after which 15.8 g of spherical dry saponified product with a particle diameter of about 50 μm to about 240 μm is obtained after drying. The macropore spacer material is also washed off or otherwise removed from the microspheres. The product is then sieved and calibrated into, e.g., about 50 μm increments, to get several size ranges, e.g., about 50 μm-100 μm, about 100 μm-150 μm, about 150 μm-210 μm. The sieved products can then be lyophilized.

### Example 7

### Administration of Bimodal Porous Micropsheres for Islet Cell Transplantation in Human Subject

An islet cell transplantation procedure is described as follows. Patients having C-peptide-negative type 1 diabetes for more than 5 years are included in the study on the basis of poor glycemic control, which is complicated by recurrent hypoglycemia or metabolic lability despite compliance with optimal medical therapy. Patients receive steroid-free immunosuppression therapy, which commences immediately prior to islet cell transplantation, with a regimen of five doses of daclizumab at a dose of mg/kg are administered i.v. over a period of 8 weeks after each transplantation. Strolimus is administered once daily to achieve a target therapeutic range of 12 to 15 ng/ml for three months after transplantation, after which the target trough range is lowered to 7 to 12 ng/ml. Tacrolimus is also administered twice daily and adjusted to achieve a target trough level of 3 to 6 ng/ml. In addition, patients are given a standard prophylactic antibiotics prior to commencement of the procedure.

All patients are sedated with intravenous midazolam and fentanyl. Oxygen is administered via the nasal cannula at 6 L/min. A right-sided percutaneous approach is used with patients positioned supine. The point of hepatic puncture (anterior or midaxillary line) is determined by using fluoroscopy, ultrasonography, or a combination of both. Total fluoroscopic time is recorded for each procedure. The subcutaneous tissues and hepatic capsule are infiltrated with local anesthetic. A 22-gauge Chiba needle is advanced into a branch of the right portal vein with fluoroscopic or US guidance. A second- or third-order branch is selected in most cases. An 18-gauge guidewire is then advanced into the main portal vein.

Islet cells are prepared essentially as described in Owen et al. (2003) Radial. 229:165-170. Briefly, pancreas organs are obtained from brain-dead donors after informed consent is received from relatives. The islet cells are isolated using a combination of enzymatic and mechanical dissolution and are prepared in xenoprotein-free medium. Islet cell transplantation proceeds if more than about 4000 purified islet cell equivalents are prepared and packed in cell volume of less than 10 ml, if ABO blood group compatibility matches, and if Gram stain was negative and endotoxin content is less than 5 endotoxin units/kg. The quantification of islet cells expressed in terms of islet cell equivalents accounts for variation in islet cell volume, with a standard islet cell measuring 150 μm.

The islet cells are administered to the patients following one of the treatment regimes:

Group I—islet cells with bimodal porous microspheres.

Group II—islet cells alone.

Group III—microspheres alone.

Islet cell preparation is suspended in 120 mL of supplemented media (M199; Mediatech, Herndon, Va.) that contained heparin, 20% human albumin, and microspheres (in Group I). Heparin (35 U/kg) is added when the packed islet cell volume is less than 5 mL, and the amount of heparin is increased to 70 U/kg if the packed cell volume exceeded 5 mL. The islet cells are administered either through this sheath or through a 5-F Kumpe catheter placed in the portal vein. Alternatively, a specially designed stiffened micropuncture set can be used with a 4-F sheath designed to accept a 0.038-inch guidewire. The tract is embolized with gelatin sponge particles. Once the stiffened micropuncture kit is available, tract embolization is no longer performed routinely.

The islet cells with (Group I) or without (Group II) microspheres, or microspheres alone (Group III) are initially administered over approximately 10 minutes using a 60-mL syringe. Portal venous pressure is recorded after the first 50-mL aliquot and after subsequent 50-mL aliquots. Alternatively, a gravity-based closed infusion bag system can be used to minimize the shear forces on the islet cells to provide an alternative indirect method of continuous portal venous pressure monitoring and to reduce the risk of preparation contamination during islet cell delivery. The procedure is terminated if portal venous pressure is higher than 20 mm Hg at the outset or if it increased to twice the baseline value or to higher than 22 mm Hg during the procedure. The patients can undergo several time of infusions, depending on each individual's needs.

Following the infusion procedure, optionally, the catheter is removed with embolization of the tract by using gelatin sponge pledgets. Complications are documented from data recorded at the time of the procedure, at clinical follow-up, and at confirmatory review of the radiology chart and images. Subjects will be evaluated for a reduction in the need for insulin, levels of fasting glucose and glycated hemoglobin, basal C-peptide secretion, and the mean amplitude of glycemic excursions over time.

### Example 8

### Administration of Bimodal Porous Microspheres in a Rat Model of Heart Damage

Preparation of Bmc: Adult Sprague-Dawley Rats are Anesthetized with i.m. administration of ketamine hydrochloride (22 mg/kg) followed by an i.p. injection of sodium pentobarbital (30 mg/kg). Under general anesthesia, bone marrow is aspirated from the tibia with a syringe containing 1 ml heparin with an 18G needle. The marrow cells are transferred to a sterile tube and mixed with 10 ml culture medium (IMDM with 10% FBS, 100 U/ml penG) and 100 μg/ml streptomycin. The tube is centrifuged at 2000 rpm for 5 min. and the cell pellet is resuspended with 5 ml culture medium. To separate bone marrow cells (BMC) and red blood cells (RBC), the gradient centrifugation method described by Yablonka-Reuveni and Nameroff is used ((1987) Histochem. 87:27-38). The cell suspension is loaded on 20% to 60% gradient of Percoll, and the cells are centrifuges at 14,000 rpm for 10 min. The top two thirds of the total volume containing most of the BMC are transferred to a tube. The cells are centrifuged at 2,000 rpm for 10 min. and then washed with PBS to remove the Percoll. This is repeated and the cell pellet is resuspended in culture medium and used for in vivo studies.

Mycardial Scar Generation: Under general anesthesia, the rats were intubated and positive pressure ventilation (180 mL/min.) is maintained with room air supplemented with oxygen (2 L/min.) using a ventilator. The rat heart is exposed through a 2 cm left lateral thoracotomy. Cryoinjury is produced with a metal probe (8×10 mm in diameter) cooled to −190° C. by immersion in liquid nitrogen and applied to the left ventrical free wall for 15 sec. This procedure was repeated 5 times and then applied for a total of 10 times with each lasting 1 min. The muscle layer and skin incision were closed with silk sutures. The rates are monitored for 4 hours postoperatively and penicillin is given by i.m. administration.

Transplantation: Three weeks after myocardial damage, the rats are randomly divided in three groups:

Group I—BMC with bimodal porous microspheres.

Group II—BMC alone.

Group III—microspheres alone.

The rat heart is exposed through a midline sternotomy under general anesthesia. Microspheres comprising varying concentrations of BMC (such as 10⁶) cells (Group I), fifty microliters of BMC suspension containing 10⁶ cells (Group II) or microspheres alone (Group III) are injected using a tuberculin syringe or other suitable catheter into the center of the left ventricular free wall scar tissue of each animal in the respective transplant groups. The chest is closed with silk sutures, and antibiotics and analgesics are given.

Heart Function Measurements: Five weeks after transplantation, the rats are anesthetized with ketamine and pentobarbital. A midline sternotomy is performed, the heart is removed and the animals are euthanized by exsanguinations. Heart function of the three groups is measured using a Langendorff apparatus and filtered Krebs-Henseleit buffer (in mmol/L: NaCl, 118; KCl, 4.7; KH₂PO₄, 1.2; CaCl₂, 2.5; MgSO₄ 1.2; NaHCO₃, 25; and glucose, 11; pH 7.4) at the pressure of 100 mm Hg equilibrated with 5% CO₂ and 95% O₂. A latex balloon is passed into the left ventricle through the mitral valve and connected to a pressure transducer, a transducer amplifier, and differentiator amplifier. After 10 min. stabilization, the coronary flow of the heart is measures in triplicate by timed collection in the empty beating state without pacing. The balloon size is increased by the addition of water in 20 μl increments from 40 μl until the left ventrical end-diastolic pressure reaches 30 mm Hg. The systolic and diastolic pressures are recorded at each balloon volume and developed pressure is calculated. The heart is weighed and its size is measured by water displacement.

Planimetry: The scar size of left ventricular free wall is measured by the techniques of Pfeffer et al. (1991) Am. J. Physiol. 260:H1406-H1414 and Jugdutt and Khan. (1994) Circulation 89:2297-2307. Briefly, the hearts are fixed in distention (30 mm Hg) with 10% neutralized formalin and then cut into slices 3 mm thick. For each section, the outer and inner lines of the left ventricle are traced onto a transparency and quantified using computed planimetry (Jandal Scientific Sigma-Scan).

Histological Studies: Tissue samples (0.5 cm³) at the transplantation site are collected at 5 weeks after transplantation and fixed in neutralized 10% formaldehyde for histological study. The samples are embedded and cut to yield 10-μm thick sections, which are stained with hematoxylin and eosin as described in the manufacturer's specifications (Sigma Chemical Co).

Identification of Transplanted BMCs in the Scar: Under general anesthesia, 4 rats are scarred and 2 weeks later bone marrow is aspirated. The BMCs are cultured and induced with 5-aza as described above. To identify the transplanted cells in the scar tissue, the cells are labeled with bromodeoxyuridine (BrdU, Sigma). Briefly, 10 μL of BrdU solution (BrdU, 50 mg; dimethyl sulfoxide, 0.8 mL; water, 1.2 mL) is added into each culture dish on the sixth day of culture and incubated with the cells for 24 hours. The labeled cells +/−microspheres are transplanted into the scar at 3 weeks after myocardial injury, and samples are collected at 5 weeks after transplantation as previously described. Monoclonal antibodies against BrdU are used to localize the transplanted bone marrow cells. Briefly, samples are serially rehydrated with 100%, 95%, and 70% ethanol after deparaffinization with toluene. Endogenous peroxidase in the sample is blocked using 3% hydrogen peroxide for 10 minutes at room temperature. The sample is treated with pepsin for 5 minutes at 42° C. and 2N HCl for 30 minutes at room temperature. After rinsing with PBS 3 times, the sample is incubated with antibodies against BrdU in a moist chamber for 16 hours at room temperature. Negative control samples are incubated in PBS (without the primary antibodies) under the same conditions. The test and control samples are rinsed with PBS 3 times (15 minutes each) and then incubated with goat anti-rabbit immunoglobulin G conjugated with peroxidase at 37° C. for 45 minutes. The samples are washed 3 times (15 minutes each) with PBS and then immersed in diaminobenzidine H₂O₂ (2 mg/mL diaminobenzidine, 0.03% H₂O₂ in 0.02 mL/L phosphate buffer) solution for 15 minutes. After washing with PBS, the samples are coverslipped and photographed.

Measurement of Capillary Density in the Scar: The number of capillary vessels is counted in the scar tissue of all groups, using a light microscope at a ×400 magnification. Five high-power fields in each scar are randomly selected, and the number of capillaries in each is averaged and expressed as the number of capillary vessels per high-power field (0.2 mm²).

### Example 9

### Administration of Bimodal Microspheres During Liver Transplantation in a Rabbit Cirrhosis Model

Male New Zealand white rabbits are randomly divided into the following groups.

Group I (control): Subcutaneous injection of olive oil twice weekly at a dosage of 0.3 ml/kg body weight for the first 2 weeks and 0.2 ml/kg thereafter until the end of 12 weeks. Portal perfusion with normal saline administered at week 13 twice weekly for another 12 weeks.

Group II: Subcutaneous injection of olive oil twice weekly at a dosage of 0.3 ml/kg body weight for the first 2 weeks and 0.2 ml/kg thereafter until the end of 12 weeks. Portal perfusion with bone marrow cells alone administered at week 13 twice weekly for another 12 weeks.

Group III: Subcutaneous injection of olive oil twice weekly at a dosage of 0.3 ml/kg body weight for the first 2 weeks and 0.2 ml/kg thereafter until the end of 12 weeks. Portal perfusion with bone marrow cells plus bimodal porous microspheres administered at week 13 twice weekly for another 12 weeks.

Group IV: Subcutaneous injection of 50% CCl₄ in olive oil twice weekly at a dosage of 0.3 ml/kg body weight for the first 2 weeks and 0.2 ml/kg body weight for another 12 weeks. Portal perfusion with normal saline administered at week 13 twice weekly for another 12 weeks.

Group V: Administration of 50% CCl₄ as in Group IV. Portal perfusion with bone marrow cells alone Administered at week 13 twice weekly for another 12 weeks.

Group VI: Administration of 50% CCl₄ as in Group IV. Portal perfusion with bone marrow cells plus bimodal porous microspheres administered at week 13 twice weekly for another 12 weeks.

All rabbits are subjected to surgery for permanent portal catheterization 2-9 days prior to portal perfusion. Under anesthesia, the rabbits are shaved of the hair coat with a razor and disinfected. Surgery is performed through an upper abdominal mid-line incision of 5 cm length. A liver sample is taken for pathological examination and hydroxyproline assay. Subsequently, the jejunal mesenteric vein is uncovered and an incision is made after isolation of a terminal branch of the vein. The distal end of the vein is ligated, and a disposable sterile epidural anesthetic catheter with a sealed tip and three distal side-holes is filled with normal saline containing heparin. The catheter is threaded through the vessel incision into the proximal vein towards the liver, and when sufficient length of catheter is in the vein, it is fixed to the vessel by ligation. The syringe is taken off and the catheter is cut into a suitable length after ensuring the catheter is in the right position. The open end of the catheter is attached to a connector with an injection cap immediately after cut down. The abdominal cavity is closed by suture, and the open end of the catheter with the injection cap filled with heparin saline is embedded subcutaneously.

Approximately 1-5×10⁷ cells of the donor mouse bone marrow cells (with or without microspheres) or saline are given to the rabbits via portal perfusion by puncturing the injection cap. The perfusion rate was set at about 2-5 ml/min. Rabbits are killed 24 h after the cell administration. Blood samples are taken from the ear margin vein prior to and post perfusion for routine blood, liver function, and renal function tests. After killing, the liver, myocardium, kidney, lung, and brain are sampled and fixed formaldehyde for histological examination.

The embodiments of the present invention described above are intended to be merely exemplary, and those skilled in the art will recognize, or be able to ascertain using no more than routine experimentation, numerous equivalents to the specific procedures described herein. All such equivalents are considered to be within the scope of the present invention and are covered by the following claims. Furthermore, as used in this specification and claims, the singular forms “a,” “an” and “the” include plural forms unless the content clearly dictates otherwise. Thus, for example, reference to “an additive” includes a mixture of two or more such additives. Additionally, ordinarily skilled artisans will recognize that operational sequence must be set forth in some specific order for the purpose of explanation and claiming, but the present invention contemplates various changes beyond such specific order.

1. A method for preparing a bimodal porous polymer particle, said method comprising:
(a) providing a homogeneous solution comprising a base polymer, a first solvent and a second solvent;
(b) adding a macropore spacer material to the solution;
(c) injecting droplets of the solution into a quenching device;
(d) quenching droplets of the solution to solidify the base polymer into particles having macropores and micropores;
(e) extracting substantially spherical particles from the quenching device, and optionally sieving the particles; and
(f) optionally washing the macropore spacer material from the particles.

2. (canceled)

3. (canceled)

4. The method of claim 1, wherein the base polymer is soluble in the first solvent.

5. The method of claim 1, wherein the base polymer is not soluble in the second solvent.

6. The method of claim 1, wherein the second solvent is miscible in the first solvent.

7. The method of claim 1, wherein the first and second solvents are miscible, such that the base polymer is substantially dissolved in the homogenous solution.

8. The method of claim 1, wherein the volume ratio of the first solvent to the total volume of the first and second solvents is between about 1% to about 50% v/v, between about 1% to about 40% v/v, between about 2% to about 30% v/v, between about 4% to about 25% v/v, or between about 5% to about 15% v/v.

9. The method of claim 1, wherein the concentration of the base polymer in the solution is between about 0.1% to about 50% by weight, between about 1% to about 40% be weight, between about 5% to about 23% by weight, or between about 10% to about 20% by weight.

10. The method of claim 1, wherein the macropore spacer material is immiscible or only slightly miscible in the first solvent and base polymer.

11. The method of claim 1, wherein the macropore spacer material is miscible in the second solvent.

12. The method of claim 1, wherein the macropore spacer material is sodium chloride.

13. The method of claim 1, wherein the second solvent is water.

14. The method of claim 1, wherein the macropore spacer material is not miscible in either the first or second solvents.

15. The method of claim 1, wherein the macropore spacer material is soluble in a third solvent or other medium used in the washing.

16. The method of claim 1, wherein the macropore spacer material is an additive.

17. The method of claim 16, wherein the macropore spacer material is cisplatin.

18. The method of claim 15, wherein the macropore spacer material is cisplatin and the medium is a nitrogen-based medium.

19. (canceled)

20. (canceled)

21. (canceled)

22. (canceled)

23. The method of claim 1, wherein the macropore spacer material is selected from the group consisting of an alkali metal and alkaline earth metal halides, phosphates, sulfates; sugars, crystals of sugars; water soluble polymers, microspheres, nanoparticles, microspheres of water-soluble polymers; proteins, albumin, and sodium chloride.

24. The method of claim 1, wherein the base polymer is soluble is in the first solvent and miscible in the second solvent, and wherein the ratio of the first solvent to the second solvent is in a range to allow the base polymer to dissolve to form a homogenous solution.

25. The method of claim 1, wherein the first solvent is 1,4-dioxane.

26. The method of claim 1, wherein the second solvent is water.

27. The method of claim 1, wherein the quenching is at a rate effective to result in crystallization of the first solvent before the onset of liquid-liquid demixing of the first and second solvent.

28. (canceled)

29. (canceled)

30. (canceled)

31. (canceled)

32. The method of claim 1, wherein the washing comprises one or more of leaching, application of heat, and sublimation.

33. The method of claim 1, further comprising providing an additive.

34. The method of claim 33, wherein the additive is provided to the solution prior to said quenching.

35. The method of claim 33, wherein the additive is provided to the solution after said quenching.

36. The method of claim 33, wherein the additive is provided to the solution during said quenching.

37. The method of claim 33, wherein the additive is provided to the solution prior to said injecting.

38. The method of claim 33, wherein the additive is provided to the solution after said injecting.

39. The method of claim 33, wherein the additive is provided to the solution during said injecting.

40. The method of claim 33, wherein the additive is provided to the solution prior to said washing.

41. The method of claim 33, wherein the additive is provided to the solution after said washing.

42. The method of claim 33, wherein the additive is provided to the solution during said washing.

43. The method of claim 33, wherein the additive is covalently attached to the polymer.

44. (canceled)

45. The method of claim 33, wherein the additive is incorporated into or coated onto the particle.

46. The method of claim 33, wherein the additive adheres to a surface of the particle.

47. The method of claim 46, wherein the additive adheres to the surface of the particle by cross-linking with the base polymer, ionic bonding, acid base reactions, receptor site attraction or gravitational forces.

48. (canceled)

49. (canceled)

50. The method of claim 1, wherein the base polymer comprises or consists of a biocompatible polymer or monomer.

51. The method of claim 1, wherein the base polymer comprises or consists of a bioabsorbable and/or biodegradable polymer or monomer.

52. The method of claim 1, wherein the base polymer comprises or consists of a polyhydroxy-alkanoate (PHA), poly(hydroxybutyrate) (PHB), poly(hydroxybutyrate co-hydroxyvaerate) (PVBV), alpha-hydroxycarboxilic and copolymers thereof, poly(lactic) acid (PLA), poly(gloycolide) (PGA); polycaprolactone (PCL); polyesteramid; alphatic co-polyester; aromatic co-polyester; polysaccharide, starch, ligno-cellulosic material, pectin, chotosan, chitin, gum, wax; protein, lipid, casein, whey, collagen, gelatin, fibrin, glycosaminoglycans (GAGS); zein, soya, gluten, polyethylene oxide/polyethylene terephthalate or copolymer thereof; a copolymer of lactic and/or glycolic acid with hydroxy-ended flexible chains, poly(alkylene glycol), hydroxyl-terminated polyethylene oxide, polypropylene oxide, poly(oxyethylene-co-oxypropylene), polytetramethylene oxide chain, poly(oxyethylene glycol), poly(oxypropylene)-poly(oxyethylene)-glycol block copolymer, poly(oxybutylene)glycol, polycaprolactone, polyhydroxybutyrate, a copolymer of polyester, polycarbonate, polyanhydride and poly(ortho ester); bisphenol-A based polyphosphoester, poly(bisphenol-A phenylphosphate), poly(bisphenol-A ethylphosphate), poly(bisphenol-A ethylphosphonate), poly(bisphenol-A phenylphosphonate), poly[bis(2-ethoxy)hydrophosphonic terephthalate], copolymer of bisphenol-A based poly(phosphoester), tyrosine-derived diphenol monomer, polyiminocarbonate, polycarbonate, polyacrylate, polyurethane polyether, desaminotyrosyl-tyrosine (DT) ester, desaminotyrosyl tyrosine ethyl ester (DTE), desaminotyrosyl tyrosine butyl ester (DTB), desaminotyrosyl tyrosine hexyl ester (DTH), desaminotyrosyl tyrosine octyl ester (DTO), a polycarbonate, polyimino-carbonate, polyarylate, polyurethane, poly(alkylene oxide ether), poly(alkylene oxide) block copolymers polymerized from dihydroxy monomers prepared from α- and β-hydroxy acids and derivatives of tyrosine, block copolymer of polycarbonates and polyarylates with poly(alkylene oxides), polycarbonate, polyimino carbonate, polyarylate, poly(alkylene oxide) block copolymer. block copolymer of polycarbonate with poly(alkylene oxide), block copolymer of polyarylate with poly(alkylene oxide), α-hydroxycarboxylic acids, poly(capro-lactone), poly(hydroxybutyrate), polyanhydride, poly(ortho ester), polyester bisphenol-A based poly(phosphoester), polycarbonate, polyarylate, copolymer of a polycarbonate and a poly(alkylene oxide), copolymer of a polyacrylate and a poly(alkylene oxide), α-hydroxycarboxylic acid, poly(capro-lactone), poly(hydroxybutyrate), polyanhydride, poly(ortho ester), polyester bisphenol-A based poly(phosphoester), or a combination thereof.

53. The method of claim 1, where in the base polymer comprises or consists of 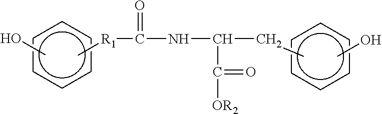
wherein R₁ is —CH═CH— or (—CH₂—)ₙ, in which n is zero or an integer from one to eight; and R₂ is selected from straight and branched alkyl and alkylaryl groups comprising up to 18 carbon atoms.

54. (canceled)

55. A bimodal porous particle comprising a base polymer, wherein the particle comprises macropores having a diameter ranging from about 20 to about 500 microns and micropores having a diameter ranging from about 1 to about 70 microns, and wherein the microspheres have a diameter ranging from about 50 to about 1100 microns.

56. (canceled)

57. (canceled)

58. The particle of claim 55, wherein the particle further comprises an additive.

59. The particle of claim 58, wherein the additive is covalently attached to the polymer.

60. (canceled)

61. The particle of claim 58, wherein the additive is incorporated into or coated onto the particle.

62. The particle of claim 58, wherein the additive adheres to a surface of the particle.

63. The particle of claim 62, wherein the additive adheres to the surface of the particle by cross-linking with the base polymer, ionic bonding, acid base reactions, receptor site attraction or gravitational forces.

64. The particle of claim 55, wherein the particle comprises or consists of a biocompatible polymer or monomer.

65. The particle of claim 55, wherein the polymer or monomer is a bioabsorbable and/or biodegradable polymer or monomer.

66. The particle of claim 55, wherein the base polymer comprises or consists of a polyhydroxy-alkanoate (PHA), poly(hydroxybutyrate) (PHB), poly(hydroxybutyrate co-hydroxyvaerate) (PVBV), alpha-hydroxycarboxilic and copolymers thereof, poly(lactic) acid (PLA), poly(gloycolide) (PGA); polycaprolactone (PCL); polyesteramid; alphatic co-polyester; aromatic co-polyester; polysaccharide, starch, ligno-cellulosic material, pectin, chotosan, chitin, gum, wax; protein, lipid, casein, whey, collagen, gelatin, fibrin, glycosaminoglycans (GAGS); zein, soya, gluten, polyethylene oxide/polyethylene terephthalate or copolymer thereof; a copolymer of lactic and/or glycolic acid with hydroxy-ended flexible chains, poly(alkylene glycol), hydroxyl-terminated polyethylene oxide, polypropylene oxide, poly(oxyethylene-co-oxypropylene), polytetramethylene oxide chain, poly(oxyethylene glycol), poly(oxypropylene)-poly(oxyethylene)-glycol block copolymer, poly(oxybutylene)glycol, polycaprolactone, polyhydroxybutyrate, a copolymer of polyester, polycarbonate, polyanhydride and poly(ortho ester); bisphenol-A based polyphosphoester, poly(bisphenol-A phenylphosphate), poly(bisphenol-A ethylphosphate), poly(bisphenol-A ethylphosphonate), poly(bisphenol-A phenylphosphonate), poly[bis(2-ethoxy)hydrophosphonic terephthalate], copolymer of bisphenol-A based poly(phosphoester), tyrosine-derived diphenol monomer, polyiminocarbonate, polycarbonate, polyacrylate, polyurethane polyether, desaminotyrosyl-tyrosine (DT) ester, desaminotyrosyl tyrosine ethyl ester (DTE), desaminotyrosyl tyrosine butyl ester (DTB), desaminotyrosyl tyrosine hexyl ester (DTH), desaminotyrosyl tyrosine octyl ester (DTO), a polycarbonate, polyimino-carbonate, polyarylate, polyurethane, poly(alkylene oxide ether), poly(alkylene oxide) block copolymers polymerized from dihydroxy monomers prepared from α- and β-hydroxy acids and derivatives of tyrosine, block copolymer of polycarbonates and polyarylates with poly(alkylene oxides), polycarbonate, polyimino carbonate, polyarylate, poly(alkylene oxide) block copolymer. block copolymer of polycarbonate with poly(alkylene oxide), block copolymer of polyarylate with poly(alkylene oxide), α-hydroxycarboxylic acids, poly(capro-lactone), poly(hydroxybutyrate), polyanhydride, poly(ortho ester), polyester bisphenol-A based poly(phosphoester), polycarbonate, polyarylate, copolymer of a polycarbonate and a poly(alkylene oxide), copolymer of a polyacrylate and a poly(alkylene oxide), α-hydroxycarboxylic acid, poly(capro-lactone), poly(hydroxybutyrate), polyanhydride, poly(ortho ester), polyester bisphenol-A based poly(phosphoester), or a combination thereof.

67. The particle of claim 55, where in the base polymer comprises or consists of 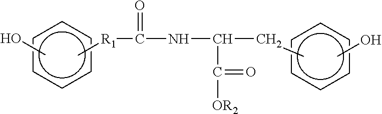
wherein R₁ is —CH═CH— or (—CH₂—)ₙ, in which n is zero or an integer from one to eight; and R₂ is selected from straight and branched alkyl and alkylaryl groups comprising up to 18 carbon atoms.

68. The particle of claim 55, wherein the particle comprises a polyvinyl alcohol.

69. (canceled)

70. The particle of claim 55, wherein the particle comprises an acrylic, acrylamide or acrylate polymer or copolymer.

71. (canceled)

72. (canceled)

73. (canceled)

74. (canceled)

75. (canceled)

76. (canceled)

77. (canceled)

78. (canceled)

79. The bimodal porous particle prepared by the method of claim 1.

80. The particle of claim 79, further comprising a cell.

81. (canceled)

82. (canceled)

83. An injectable composition comprising the particle of claim 79 and a pharmaceutically acceptable carrier.

84. A method of complete or partial embolization in a patient, comprising administering the particle of claim 79 to the patient.

85. A method of treating or otherwise managing a cancer or tumor, comprising administering the particle of claim 79 to a blood vessel, vein or artery of the patient that directly or indirectly supplies the cancer or tumor with blood.

86. (canceled)

87. A method for delivering cells to a patient, comprising administering the particle of claim 80 to the patient.

88. (canceled)

89. A method for retaining cells in a tissue or organ of a patient, comprising administering the particle of claim 80 to the patient, and contacting the cells with the tissue or organ.

90. A method for engrafting cells in a tissue or organ of a patient, comprising administering the particle of claim 80 to the patient, and contacting the cells with the tissue or organ.

91. A method for tissue regeneration in a patient, comprising administering the particle of claim 80 to the patient.

92. (canceled)

93. The method of claim 91, wherein the tissue is heart tissue, lung tissue, nervous tissue, brain tissue, liver tissue, pancreas tissue, uterine tissue, blood vessel, vein, artery, bone, tendon, muscle, kidney tissue, endocrine tissue, solid organ tissue, or a wound or other injured tissue.

94. (canceled)

95. (canceled)

96. A method for islet cell transplantation in a patient, comprising administering the particle of claim 80 to the patient, wherein the cell is an islet cell.

97. A method for treating or otherwise managing diabetes in a patient, comprising administering the particle of claim 80 to the patient, wherein the cell is an islet cell.

98. (canceled)

99. (canceled)

100. (canceled)

101. A method for intraarterial brachytherapy in a patient, comprising administering the particle of claim 79 to the patient.

102. (canceled)

103. (canceled)

104. (canceled)

105. (canceled)

106. (canceled)

107. A method for delivering an additive to a patient, comprising administering the particle of claim 79 to the patient, wherein the particle comprises the additive.

108. (canceled)

109. (canceled)

110. (canceled)

111. A method of prolonged and/or controlled delivery of an additive in a patent, comprising administering the particle of claim 79 to the patient, wherein the particle comprises the additive.

112. (canceled)

113. (canceled)

114. (canceled)

115. A method of trapping, filtering or extracting cells from the blood of a patient, comprising administering the particle of claim 79, wherein the particle allows for perfusion through the particle, and wherein the particle traps, filters or extracts the cells from the blood of the patient.

116. An apparatus for fabricating bimodal porous polymer particles, comprising:
(a) a storage vessel;
(b) a quenching tower;
(c) an injector comprising a nozzle, wherein the nozzle has a diameter ranging from about 5 to about 1100 microns in diameter; and wherein the vessel is connected to the injector; and
(d) one or more microsieves.

117. (canceled)

118. (canceled)

119. (canceled)

120. (canceled)

121. (canceled)

122. The particle of claim 55, further comprising a cell.

123. An injectable composition comprising the particle of claim 55 and a pharmaceutically acceptable carrier.

124. A method of complete or partial embolization in a patient, comprising administering the particle of claim 55 to the patient.

125. A method of treating or otherwise managing a cancer or tumor, comprising administering the particle of claim 55 to a blood vessel, vein or artery of the patient that directly or indirectly supplies the cancer or tumor with blood.

126. A method for delivering cells to a patient, comprising administering the particle of claim 122 to the patient.

127. A method for retaining cells in a tissue or organ of a patient, comprising administering the particle of claim 122 to the patient, and contacting the cells with the tissue or organ.

128. A method for engrafting cells in a tissue or organ of a patient, comprising administering the particle of claim 122 to the patient, and contacting the cells with the tissue or organ.

129. A method for tissue regeneration in a patient, comprising administering the particle of claim 122 to the patient.

130. The method of claim 129, wherein the tissue is heart tissue, lung tissue, nervous tissue, brain tissue, liver tissue, pancreas tissue, uterine tissue, blood vessel, vein, artery, bone, tendon, muscle, kidney tissue, endocrine tissue, solid organ tissue, or a wound or other injured tissue.

131. A method for islet cell transplantation in a patient, comprising administering the particle of claim 129 to the patient, wherein the cell is an islet cell.

132. A method for treating or otherwise managing diabetes in a patient, comprising administering the particle of claim 129 to the patient, wherein the cell is an islet cell.

133. A method for intraarterial brachytherapy in a patient, comprising administering the particle of claim 55 to the patient.

134. A method for delivering an additive to a patient, comprising administering the particle of claim 55 to the patient, wherein the particle comprises the additive.

135. A method of prolonged and/or controlled delivery of an additive in a patent, comprising administering the particle of claim 55 to the patient, wherein the particle comprises the additive.

136. A method of trapping, filtering or extracting cells from the blood of a patient, comprising administering the particle of claim 55, wherein the particle allows for perfusion through the particle, and wherein the particle traps, filters or extracts the cells from the blood of the patient.
